(19) United States
(12) Patent Application Publication
(10) Pub. No.: US 2026/0167936 A1
(43) Pub. Date: Jun. 18, 2026

(54) STABLE CORNEAL ENDOTHELIAL CELL (CEC) COMPOSITIONS AND METHODS OF USE

(71) Applicant: Aurion Biotech, Inc., Seattle, WA (US)

(72) Inventors: Arnaud Lacoste, Cambridge, MA (US); Jay Yasin, Brighton, MA (US); Frada Berenshteyn, Woburn, MA (US)

(21) Appl. No.: 19/417,690

(22) Filed: Dec. 12, 2025

Related U.S. Application Data
(60) Provisional application No. 63/734,026, filed on Dec. 13, 2024.

(51) Int. Cl. C12N 5/00; A61K 35/30; C12N 5/079

(52) U.S. Cl. CPC C12N 5/526; A61K 35/30; C12N 5/0621

(57) ABSTRACT

Provided are stable cell compositions comprising a population of cultured human corneal endothelial cells (CECs) and a rho kinase (ROCK) inhibitor. The stable cell composition has improved stability, e.g., at least 70% cell viability following 7 days of storage at 20 degrees Celsius. The stable cell compositions can be used in CEC therapies to treat corneal endothelial diseases.

### RELATED APPLICATIONS

This application claims the benefit of U.S. Provisional Application No. 63/734,026, filed Dec. 13, 2024, the contents of which are herein incorporated by reference in their entirety.

### BACKGROUND

Corneal endothelial dysfunction is a sight-threatening condition with significant, progressive impact. Current treatment methods are associated with a number of caveats and complications. Thus, there remains a need for improved methods and compositions useful for treatment of corneal endothelial dysfunction.

### SUMMARY

The present disclosure provides, among other things, corneal endothelial cells (CECs) and CEC compositions with improved shelf-life (e.g., maintained viability and efficacy of CECs over periods of time). In various embodiments, without wishing to be bound by any particular scientific theory, the present disclosure provides that including a rho kinase (ROCK) inhibitor in a composition comprising CECs and/or storing a CEC composition (e.g., a CEC composition comprising a ROCK inhibitor) under conditions disclosed herein may improve the shelf-life of the composition while maintaining characteristics of the CECs (e.g., characteristic cell surface markers of the CECs). The present disclosure therefore further provides, among other things, methods and uses of CEC compositions with improved shelf-life.

For therapeutic purposes, suitable compositions comprising cultured human corneal endothelial cells (CECs) must have sufficiently high viability and be able to maintain characteristics of CECs including adhesion and characteristic surface markers. This disclosure provides stable human corneal endothelial cell (CEC) compositions with sufficiently high viability over time. The stable CEC compositions described herein maintain characteristics of CECs, including adhesion and characteristic surface markers, such that the CEC cell populations can be used for therapy. The compositions can include cultured CECs from any source, preferably where the starting population of cells has the suitable characteristics, e.g., viability, adhesion, and cell surface markers so that the CECs can be used in therapies to treat corneal endothelial diseases.

Provided herein is a stable cell composition comprising: a) a population of cultured human corneal endothelial cells (CECs) comprising at least 1×10⁵ CECs; and b) a rho kinase (ROCK) inhibitor, wherein the composition has at least 70% cell viability following 7 days of storage at 20 degrees Celsius. In one embodiment, the composition has at least 70% cell viability following 10 days of storage at 20 degrees Celsius. In one embodiment, the composition has at least 70% cell viability following 14 days of storage at 20 degrees Celsius. In one embodiment, the composition has at least 70% cell viability following 21 days of storage at 20 degrees Celsius. In one embodiment, the composition has at least 70% cell viability following 28 days or more of storage at 20 degrees Celsius. In one embodiment, the composition has at least 75% cell viability following storage at 20 degrees Celsius. In another embodiment, the composition has at least 80% cell viability following storage at 20 degrees Celsius.

In a further embodiment, at least 75% of the CECs in the population have an E-ratio as assessed by immunophenotyping. In some embodiments, at least 80% of the CECs in the population have an E-ratio as assessed by immunophenotyping.

The stable cell composition described herein, in certain embodiments, comprises a population comprising at least 1×10⁶ CECs.

In one embodiment, the CECs used in the stable cell compositions described herein are obtained after at least passage 3 (P3) in culture. In another embodiment, the CECs are obtained after at least passage 4 (P4) in culture.

In one embodiment, the ROCK inhibitor in the stable cell composition is Y-27632. In one embodiment, the stable cell composition comprises about 10 μM-500 μM, 50 μM-250 μM, 80 μM-120 μM, or 100 μM of Y-27632.

In one embodiment, the stable cell composition is acceptable for intracameral administration.

In another embodiment, the stable cell composition comprises an effective dose of CECs for treating or preventing corneal endothelial cell disease in a human subject in need thereof. In one embodiment, the effective dose is about 1×10⁶ CECs per eye.

Also provided herein is a container containing the stable cell composition of the invention. In one embodiment, the CECs of the composition are not substantially adherent to the surface of the container. In one embodiment, the population of CECs in the container is about 1.70×10⁶ cells to about 5×10⁶ cells.

Also provided herein is a method of treating or preventing a corneal endothelial disease in a human subject in need thereof, said method comprising administering the stable cell composition provided herein.

In some aspects, the present disclosure provides a composition comprising a population of cultured human corneal endothelial cells and a rho kinase (ROCK) inhibitor, wherein the population of cultured human corneal endothelia cells has at least 70% viability for at least about 3 days at a first temperature, the first temperature is between about 10° C. and about 30° C., and the population of the cultured human corneal endothelial cells is in a suspension form or is not substantially adherent to a surface of a container of the composition.

In some embodiments, the composition comprises Dulbecco's modified eagle medium (DMEM); optionally wherein the composition further comprises human serum albumin (HSA). In some embodiments, the ROCK inhibitor is Y-27632, optionally wherein the composition comprises about 10 μM to about 500 μM, about 50 μM to about 250 μM, about 80 μM to about 120 μM, or about 100 μM of Y-27632; further optionally wherein the composition comprises about 100 μM of Y-27632. In some embodiments, the composition comprises about 100 μM of Y-27632, the population of cultured human corneal endothelia cells has at least 70% viability for at least 7 days at the first temperature, and the first temperature is between about 15° C. and about 25° C. In some embodiments, the population of cultured human corneal endothelia cells has at least 70% viability for about 7 days to about 20 days at the first temperature.

In some embodiments, (i) at least 75% of the population of cultured human corneal endothelial cells are CD166 positive, CD105 negative to weakly positive, and CD44 negative to weakly positive; (ii) at least 75% of the population of cultured human corneal endothelial cells are CD166 positive, CD105 negative, and CD44 negative; (iii) at least 75% of the population of cultured human corneal endothelial cells are CD24 negative, CD166 positive, CD105 negative to weakly positive, and CD44 negative to weakly positive; (iv) at least 75% of the population of cultured human corneal endothelial cells are CD24 negative, CD166 positive, CD105 negative, and CD44 negative; (v) at least 75% of the population of cultured human corneal endothelial cells are CD26 negative, CD166 positive, CD105 negative to weakly positive, and CD44 negative to weakly positive; or (vi) at least 75% of the population of cultured human corneal endothelial cells are CD26 negative, CD166 positive, CD105 negative, and CD44 negative.

In some embodiments, the composition comprises at least about 1×10⁵, at least about 5×10⁵, or at least about 1×10⁶ cultured human corneal endothelial cells; optionally wherein the composition comprises at least about 1.7×10⁶ cultured human corneal endothelial cells.

In some aspects, the present disclosure provides a method of treating a corneal endothelial disease in a human subject in need thereof, comprising administering a composition disclosed herein to the subject, wherein the composition is stored for at least 3 days at the first temperature before administration.

In some embodiments, the composition is stored for about 7 days to about 20 days at the first temperature before administration. In some embodiments, the cultured human corneal endothelial cells maintain at least 70% viability after storage at the first temperature. In some embodiments, the method comprises administering at least about 1×10⁵, at least about 5×10⁵, or at least about 1×10⁶ corneal endothelial cells to an anterior chamber of an eye of the human subject.

In some embodiments, the composition does not cause or substantially cause ocular adverse effects after administration; optionally wherein the ocular adverse effects comprise ocular hypertension, conjunctival hemorrhage, epithelial defect, ocular pain, cystoid macular edema, and/or corneal abrasion. In some embodiments, the corneal endothelial disease is selected from a bullous keratopathy, a corneal edema, a corneal leukoma, a corneal endothelial inflammation, and a corneal dystrophy; optionally wherein the corneal dystrophy is Fuchs' dystrophy.

In some aspects, the present disclosure provides a vial comprising a composition disclosed herein.

In some aspects, the present disclosure provides a method of improving shelf-life of a composition comprising corneal endothelial cells, the method comprising storing the composition at a first temperature, wherein the composition comprises a ROCK inhibitor, the first temperature is between about 10° C. and about 30° C., the corneal endothelial cells are in a suspension form or are not substantially adherent to a surface of a container of the composition, and the corneal endothelial cells maintain at least 70% viability after storage at the first temperature for at least about 3 days.

In some aspects, the present disclosure provides a method of preparing a corneal endothelial cell composition, comprising storing the corneal endothelial cell composition at a first temperature, wherein the corneal endothelial cell composition comprises corneal endothelial cells and a ROCK inhibitor, the first temperature is between about 10° C. and about 30° C., the corneal endothelial cells are in a suspension form or are not substantially adherent to a surface of a container of the composition, and the corneal endothelial cells maintain at least 70% viability after storage at the first temperature for at least about 3 days.

In some embodiments, the composition comprises Dulbecco's modified eagle medium (DMEM); optionally wherein the composition further comprises human serum albumin (HSA). In some embodiments, the ROCK inhibitor is Y-27632, optionally wherein the composition comprises about 10 μM to about 500 μM, about 50 μM to about 250 μM, about 80 μM to about 120 μM, or about 100 μM of Y-27632; further optionally wherein the composition comprises about 100 μM of Y-27632. In some embodiments, the composition comprises about 100 μM of Y-27632, the corneal endothelial cells maintain at least 70% viability after storage at the first temperature for at least about 7 days, and the first temperature is between about 15° C. and about 25° C.

In some embodiments, (i) at least 75% of the corneal endothelial cells are CD166 positive, CD105 negative to weakly positive, and CD44 negative to weakly positive; (ii) at least 75% of the corneal endothelial cells are CD166 positive, CD105 negative, and CD44 negative; (iii) at least 75% of the corneal endothelial cells are CD24 negative, CD166 positive, CD105 negative to weakly positive, and CD44 negative to weakly positive; (iv) at least 75% of the corneal endothelial cells are CD24 negative, CD166 positive, CD105 negative, and CD44 negative; (v) at least 75% of the corneal endothelial cells are CD26 negative, CD166 positive, CD105 negative to weakly positive, and CD44 negative to weakly positive; or (vi) at least 75% of the corneal endothelial cells are CD26 negative, CD166 positive, CD105 negative, and CD44 negative.

In some embodiments, the corneal endothelial cells are allogeneic human corneal endothelial cells, and the corneal endothelial cells are cultured prior to storing.

In some embodiments, the composition comprises at least about 1×10⁵, at least about 5×10⁵, or at least about 1×10⁶ corneal endothelial cells; optionally wherein the composition comprises at least about 1.7×10⁶ corneal endothelial cells.

### BRIEF DESCRIPTION OF THE DRAWINGS

The Drawings included herein are for illustration purposes only and not for limitation.

FIG. 1 provides a table showing stability of human corneal endothelial cells in formulations with or without Y-27632.

### DETAILED DESCRIPTION

The present disclosure provides, among other things, CECs and CEC compositions having improved shelf-life. The present disclosure also provides, among other things, methods and uses of the CECs and CEC compositions disclosed herein.

### Definitions

In order for the present disclosure to be more readily understood, certain terms are first defined below. Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

As used herein, “a”, “an”, and “the” refer to one or to more than one (i.e., to at least one) of the grammatical object of the article. By way of example, “an element” discloses embodiments of exactly one element and embodiments including more than one element.

As used herein, the term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

As used herein, the term “administration” or “administering” typically refers to administration of a composition to a subject or system to achieve delivery of an agent that is, or is included in, the composition.

Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact (e.g., covalently or non-covalently), directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, or a combination thereof.

As used herein, the term “between” refers to content that falls between indicated upper and lower, or first and second, boundaries (or “bounds”), inclusive of the boundaries. Similarly, the term “from”, when used in the context of a range of values, indicates that the range includes content that falls between indicated upper and lower, or first and second, boundaries, inclusive of the boundaries.

As used herein, the term “corneal endothelial cell” or “CEC” refers to a cell from a corneal endothelium layer. Preferably, CECs are human CECs. Included in the term are CECs obtained from a donor corneal endothelium layer.

As used herein, a cell derived from corneal endothelial tissue is referred to as “corneal endothelial tissue derived cell.” A cell that becomes a corneal endothelial cell by differentiation is referred to as a “corneal endothelial progenitor cell.”

As used herein, the term “corneal endothelial disease” refers to diseases affecting corneal endothelial cells. Non-limiting examples of corneal endothelial diseases include bullous keratopathy, corneal endothelial dystrophies (e.g., cornea guttata, Fuchs endothelial corneal dystrophy, posterior polymorphous corneal dystrophy, and congenital hereditary corneal endothelial dystrophy), iridocorneal endothelial syndrome, viral diseases (e.g., cytomegalovirus endotheliitis and herpetic endotheliitis), exfoliation syndrome, and corneal endothelial graft rejection; as well as inflammation or physical damage associated with external factors, such as keratouveitis, interstitial keratitis, corneal endotheliitis, corneal endothelial cell loss after corneal transplantation, corneal injury after intraocular surgery (e.g., cataract surgery, vitreous surgery, glaucoma surgery), corneal injury induced by glaucomatous attack, corneal injury caused by long-term contact lens use, corneal trauma, corneal edema, and intrapartum corneal trauma.

As used herein, the term “cultured” refers to cells that have been maintained, propagated, expanded, passaged, and/or otherwise manipulated outside a living organism in an artificial environment (e.g., in vitro or ex vivo) for any period sufficient to preserve viability and/or alter number, phenotype, or functional state. “Cultured” cells include primary cells isolated from donor tissue and maintained in medium; cells expanded through one or more passages; cells conditioned by exposure to defined medium components (e.g., serum, serum-free supplements, growth factors, ROCK inhibitors), extracellular matrix coatings, feeder layers, or co-culture; and cells collected or harvested following culture (e.g., post-culture harvest from a vessel or bioreactor). The term “cultured” is not limited by passage number, seeding density, vessel type, substrate, oxygen tension, or duration of maintenance, and does not require that the cells actively divide.

As used herein, the term “expanding” refers to a cell culture process that includes transferring (i.e., passaging) cells into a new culture vessel at a lower cell density in order to permit further growth of the cells.

As used herein, “expression” refers individually and/or cumulatively to one or more biological process that result in production from a nucleic acid sequence of an encoded agent (i.e., an expression product), such as an RNA and/or a polypeptide. Expression specifically includes either or both of transcription and translation. A nucleic acid or cell that produces the encoded agent can be said to express the encoded agent.

As used herein, the terms “improve”, “increase”, “inhibit”, “decrease” and “reduce”, and grammatical equivalents thereof, indicate qualitative or quantitative difference from a reference.

As used here, the term “or” is understood as “and/or” and includes both inclusive and exclusive use of the term unless clearly indicated otherwise by context.

As used herein, the term “passage”, as used herein in the context of cell culture, refers to the removal of medium and transfer of cells from a previous culture into fresh medium. Passaging allows for a lower cell density that can stimulate further propagation. Passaging of cells is also referred to as subculturing. When used in reference to a number, a passage number, e.g., P1, indicates how many times the cell population has been passaged. P0 indicates an initial cell culture.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

As used herein, the term “sample” typically refers to an aliquot of material obtained or derived from a source of interest. In some embodiments, a source of interest is a biological or environmental source. In some embodiments, a sample is a “primary sample” obtained directly from a source of interest. In some embodiments, the term “sample” refers to a preparation that is obtained by processing of a primary sample (e.g., by removing one or more components of and/or by adding one or more agents to a primary sample). Such a “processed sample” can include, for example cells, nucleic acids, or proteins extracted from a sample or obtained by subjecting a primary sample to techniques such as isolation and/or purification of certain components.

As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. In some instances, a human subject can be interchangeably referred to as a “patient” or “individual.” A subject administered an agent associated with treatment of a disease, disorder, or condition with which the subject is associated can be referred to as a subject in need of the agent, i.e., as a subject in need thereof.

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

### Stable Corneal Endothelial Cell (CEC) Compositions

In humans, the cornea is comprised of five layers, in order from the outside (body surface) to the inside, corneal epithelium, Bowman's membrane (external boundary), Lamina propria, Descemet's membrane (internal boundary), and corneal endothelium. Unless specifically noted otherwise, parts other than epithelium and endothelium may be collectively called corneal stroma. The corneal endothelium is composed of a single layer of cells located on the posterior surface of the cornea, facing the anterior chamber. The endothelium governs fluid and nutrient transport across the posterior surface of the cornea in a pump-and-barrier function and maintains the cornea in an optimized state required for optical transparency and optimal vision.

Unlike the epithelium, which has a self-renewing capacity, the endothelium is not capable of regenerating. Corneal endothelial disease is a sight-threatening and debilitating condition affecting millions of people throughout the world. Corneal transplants can be used to treat corneal disease, but often have variable results, as well as being limited by the number of available donors in comparison to the number of patients in need.

Injectable cell therapies to treat corneal endothelial disease have recently been developed. Human donor corneas are used to obtain CECs suitable for injection into a patient. CECs are first isolated from donor cornea and subsequently cultured and expanded in media. There is, however, a narrow window from the cell culture to transplantation.

CEC therapy provides a method to treat corneal endothelial disease in far more patients than would be possible by corneal transplant, where each donor cornea can only be used for treatment of one eye. CEC therapy relies on culturing and expansion of primary human CECs, allowing, e.g., up to 1000 patients to be treated from cells initially provided from a single donor cornea. CEC therapy is limited, however, by the ability to provide suitable CEC populations to patients in a timely manner for treatment.

In various embodiments, provided herein are compositions of stable CEC populations for use in therapy. The stable compositions described herein have improved shelf-life, while maintaining the quality necessary for therapeutic use in humans.

### Corneal Endothelial Cells (CECs)

In various embodiments, the disclosure provides CECs, e.g., CECs suitable for use in therapy (e.g., for use in treatment of corneal endothelial disease).

In various embodiments, the CECs of the present disclosure are cultured (e.g., expanded, differentiated, and/or matured) according to methods disclosed herein prior to use, e.g., use in therapy (e.g., in treatment of a corneal endothelial disease). In various embodiments, the CECs of the present disclosure are stored in compositions (e.g., compositions disclosed herein or prepared according to methods disclosed herein) prior to use, e.g., use in therapy (e.g., in treatment of corneal endothelial disease). In various embodiments, the CECs of the present disclosure are cultured (e.g., expanded, differentiated, and/or matured) according to methods disclosed herein and stored in compositions (e.g., compositions disclosed herein or prepared according to methods disclosed herein) prior to use, e.g., use in therapy (e.g., in treatment of corneal endothelial disease).

In various embodiments, the CECs of the present disclosure are functional mature differentiated CECs. In various embodiments, the CECs are capable of eliciting a human corneal endothelial functional property when administered into an anterior chamber of a human eye. In various embodiments, a functional mature differentiated CEC of the present disclosure refers to a mature and differentiated CEC having a corneal endothelial functional property. In various embodiments, a corneal endothelial functional property refers to a functional property that a mature differentiated cornea or corneal cell has in a normal state (e.g., in the state of a native corneal endothelial cell).

In various embodiments, a corneal endothelial functional property of a cell can be confirmed from, e.g., the cell forming a small hexagonal cobblestone shape and using an energy metabolism system by mitochondrial function. In various embodiments, a corneal endothelial functional property can be determined by a surrogate marker as an indicator. In some embodiments, determination can be made with any one of or any combination of surrogate markers selected from (1) retention of endothelial pumping/barrier functions (including claudin expression), (2) adhesion/attachment to a specific laminin, (3) secreted cytokine profile, (4) produced micro RNA (miRNA) profile, (5) produced metabolite profile, (6) saturated cell density upon in vitro culture, and (7) spatial size and distribution of cells obtained in culturing. In various embodiments, retention of endothelial pumping/barrier functions can be determined using a pumping function measuring method or a barrier function measuring method commonly used for corneal endothelia. Examples include techniques described in Wigham C, Hodson S.: Current Eye Research, 1, 37-41, 1981; Hodson S, Wigham C.: J Physiol., 342:409-419, 1983; Hatou S., Yamada M., Akune Y., Mochizuki H., Shiraishi A., Joko T., Nishida T., Tsubota K.: Investigative Ophthalmology & Visual Science, 51, 3935-3942, 2010. Claudin expression can be confirmed by using a known approach in the art such as an immunological approach. In various embodiments, adhesion/attachment to a specific laminin can be determined via measurement of adhesion to laminin 511 (composite of alpha5 chain, beta1 chain, and gamma1 chain), laminin 521 (composite of alpha5 chain, beta2 chain, and gamma1 chain), or a functional fragment thereof (e.g., laminin 511-E8 fragment) and/or increase in integrin (e.g., alpha3beta1, alpha6beta1 or the like) expression. Alpha5 chain (LAMA5) is a subunit of a protein (laminin) of a cell adhesion molecule in an extracellular matrix, and is called LAMA5, KIAA1907, or the like. For human LAMA5, the sequences of the gene and protein can be found through NCBI registration numbers NM_005560 and NP_005551, respectively. Beta1 chain (LAMB1) is a subunit of a protein (laminin) of a cell adhesion molecule in an extracellular matrix, and is called LAMB1, CLM, LIS5, or the like. For human LAMB1, the sequences of the gene and protein can be found through NCBI registration numbers NM_002291 and NP_002282, respectively. Beta2 chain (LAMB2) (laminin S) is a subunit of a protein (laminin) of a cell adhesion molecule in an extracellular matrix, and is called LAMB2, LAMS, NPHS5, or the like. For human LAMB2, the sequences of the gene and protein can be found through NCBI registration numbers NM_002292 and NP_002283, respectively. Gamma1 chain (LAMC1) is a subunit of a protein (laminin) of a cell adhesion molecule in an extracellular matrix, and is called LAMC1, LAMB2, or the like. For human LAMC1, the sequences of the gene and protein can be found through NCBI registration numbers NM_002293 and NP_002284, respectively. In various embodiments, secreted cytokine profiles can be determined by measuring the production level of cytokines in biological samples (e.g., serum, or anterior aqueous humor of the eye). Such cytokines include, without limitation to, RANTES, PDGF-BB, IP-10, MIP-1b, VEGF, EOTAXIN, IL-1ra, IL-6, IL-7, IL-8, IL-0, IL-10, IL-12 (p70), IL-13, IL-17, FGFbasic, G-CSF, GM-CSI, IFN-gamma, MCP-1, MIP-1a, TNF-alpha, and the like. Analysis can be performed using a cytokine measuring kit and analysis system such as Bio-Plex for integrated analysis of cytokines.

In various embodiments, the produced microRNA (miRNA) profile can be determined using analysis methods known in the art. For example, Toray's “3D-Gene” human miRNA oligochip (miRBase version 17) can be used. In various embodiments, the produced metabolite profile can be determined using analysis methods known in the art. For example, a metabolic extract of an intracellular metabolite can be prepared from a cell culture (e.g., CEC culture) sample having methanol and an internal standard reagent such as Internal Standard Solution (Human Metabolome Technologies; HMT, Inc., Tsuruoka, Japan). Capillary electrophoresis-mass spectrometry (CE-MS) analysis can be preformed to analyze the metabolite. Metabolome profile can be measured according to a method developed by Soga, et al. (Soga, D. et al., T. Soga, et al., Anal. Chem. 2002; 74:2233-2239 Anal. Chem. 2000; 72:1236-1241; T. Soga, et al., J. Proteome Res. 2003; 2:488-494) with automatic integration software (MasterHands, Keio University, Tsuruoka, Japan (M. Sugimoto, et al., Metabolomics, 2009; 6:78-95) and analyzed with MassHunter Quantitative Analysis (Agilent Technologies, Santa Clara, CA, USA). Examples of metabolites include any products related to a product of energy metabolism system in a mitochondrial system, a glutathione metabolic system product, a methionine metabolic cycle product, a lipid metabolite, a pentose phosphate pathway product, a tricarboxylic acid (TCA) cycle metabolite, or a glycolytic system metabolite, such as succinic acid (succinate), Pro, Gly, glycerol 3-phosphate, Glu, lactic acid (lactate), arginosuccinic acid (arginosuccinate), xanthine, N-carbamoyl aspartic acid (N-carbamoyl aspartate), isocitric acid (isocitrate), cis-aconitic acid (cis-aconitate), citric acid (citrate), Ala, 3-phosphoglyceric acid (3-phosphoglycerate), hydroxyproline, malic acid (malate), uric acid (urate), betaine, folic acid (folate), Gln, 2-oxoisovaleric acid (2-oxoisovalerate), pyruvic acid (pyruvate), Ser, hypoxanthine, Asn, Trp, Lys, choline, Tyr, urea, Phe, Met, carnosine, Asp, ornithine, Arg, creatine, 2-hydroxy glutaminic acid (2-hydroxy glutamate), beta-Ala, citrulline, Thr, Ile, Leu, Val, creatinine, His, or N,N-dimethyl glycine.

In various embodiments, the saturated cell density during in vitro culture can be determined by measuring the cell density under appropriate culture conditions described herein. Cell density may be measured in parallel with the cell size. In various embodiments, cell density and/or cell size may be determined using photo-taking phase contrast microscope images taken with a suitable image capturing system, e.g., a BZ X-700 Microscope (Keyence, Osaka, Japan) with an inverted microscope system (CKX41, Olympus, Tokyo, Japan). Cell density can be quantified with suitable cell counting software (e.g., BZ-H3C Hybrid cell count software (Keyence)). In various embodiments, the spatial size and distribution of cells in culture (e.g., in culture dish) can also be determined using the measurements and images of the cells.

In various embodiments, a CEC of the present disclosure (e.g., a CEC capable of eliciting a human corneal endothelial functional property) expresses a phenotype (e.g., a cell surface marker phenotype) disclosed herein. As used herein, “cell surface marker” refers to any biological material expressed on a cell surface. This may also be referred to as a cell surface antigen, surface antigen, or surface marker. A cell surface marker can be identified as an antigen binding to a monoclonal antibody. Cell surface markers referred to as CD (cluster of differentiation) markers or CD antigens in the art are also encompassed. In some embodiments, the CEC is CD166 positive. In some embodiments, the CEC is CD166 positive and CD105 negative to weakly positive. In some embodiments, the CEC is CD166 positive and CD105 negative. In some embodiments, the CEC is CD166 positive, CD105 negative to weakly positive, and CD44 negative to weakly positive. In some embodiments, the CEC is CD166 positive, CD105 negative, and CD44 negative. In some embodiments, the CEC is CD24 negative. In some embodiments, the CEC is CD26 negative. In some embodiments, the CEC is CD166 positive, CD105 negative to weakly positive, CD24 negative, and CD44 negative to weakly positive. In some embodiments, the CEC is CD166 positive, CD105 negative, CD24 negative, and CD44 negative. In various embodiments, a phenotype of CD166 positive, CD105 negative to weakly positive, CD24 negative, and CD44 negative to weakly positive, or a phenotype of CD166 positive, CD105 negative, CD24 negative, and CD44 negative, is referred to as E-ratio (effector ratio) in the present disclosure. In some embodiments, the CEC is CD166 positive, CD105 negative to weakly positive, CD26 negative, and CD44 negative to weakly positive. In some embodiments, the CEC is CD166 positive, CD105 negative, CD26 negative, and CD44 negative. In some embodiments, the CEC is CD166 positive, CD105 negative to weakly positive, CD26 negative, CD24 negative, and CD44 negative to weakly positive. In some embodiments, the CEC is CD166 positive, CD105 negative, CD26 negative, CD24 negative, and CD44 negative.

In some embodiments, the CEC is CD90 negative to weakly positive. In some embodiments, the CEC is CD90 negative to weakly positive, CD133 negative, and CD26 negative. In some embodiments, the CEC is CD90 negative to weakly positive, CD133 negative, and CD24 negative. In some embodiments, the CEC is CD90 negative to weakly positive, CD133 negative, CD26 negative, and CD24 negative. In some embodiments, the CEC is CD90 negative to weakly positive, CD133 negative, CD166 positive, CD105 negative, CD24 negative, and CD44 negative. In some embodiments, the CEC is CD90 negative to weakly positive, CD133 negative, CD166 positive, CD105 negative, CD26 negative, and CD44 negative.

The cell surface marker phenotype (e.g., the cluster of differentiation (CD) marker phenotype) may be identified via common techniques and methods in the art, such as flow cytometry analysis. Cell immunophenotyping via flow cytometry is well known in the art. The characterization of positive and negative (or weekly positive) marker profiles through gating has also been well known in the art. Gating is a fundamental aspect of flow cytometry and a skilled person understands how to gate and analyze data when immunophenotyping cells (e.g., CECs disclosed herein). For example, an initial gating (forward scatter vs. side scatter) can be performed to exclude debris. Singlet population and viability (live/dead) cells can then be determined. For the markers (e.g., the CD markers disclosed herein), the left side of the interval gate for each marker in the multicolor flow cytometry analysis can be set based on the pre-qualification Fluoresce Minus One (FMO) control for the marker. The upper limit of the FMO control's fluorescence distribution can be used to set the negative gating. Cells with fluorescence well above the FMO-defined boundary and form a clearly separate population may be considered positive. Cells in the fully stained sample that fall just above the FMO-defined negative boundary but do not form a clearly separate population may be considered weakly positive.

In various embodiments, a CEC of the present disclosure (e.g., a CEC capable of eliciting a human corneal endothelial functional property) is capable of adhering to surrounding cells. In various embodiments, the CEC expresses tight junction protein ZO-1. In various embodiments, a CEC of the present disclosure expresses Na+/K+ ATPase (ATP-dependent sodium-potassium pump). In various embodiments, a CEC of the present disclosure expresses ZO-1 and Na+/K+ ATPase.

In various embodiments, a CEC of the present disclosure is CD90 negative to weakly positive, CD133 negative, CD26 negative, LGR5 negative, SSEA3 negative, MHC1 weakly positive, MHC2 negative, PDL1 positive, ZO-1 positive, or Na+/K+ ATPase positive. In some embodiments, the CEC is CD200 negative. In various embodiments, a CEC of the present disclosure is CD105 negative to weak positive, CD24 negative, CD26 negative, LGR5 negative, SSEA3 negative, MHC1 weak positive, MHC2 negative, ZO-1 positive, Na⁺/K⁺ ATPase positive.

In various embodiments, a CEC of the present disclosure expresses at least one of sodium-potassium ATPase, ZO-1, VDAC3, SLC4A4, CLCN3, COL4A2, COL8A1, COL8A2, CDH2, CD98, CD166, CD340, Integrin α3β1, CD56, Prdx-6, CD248, SLC4A11, and CYYR1.

Measurement and quantification of polynucleotide or polypeptide expression can be accomplished by suitable methods known in the art, including, for example, northern blot, dot blot, PCR, immunohistochemistry methods such as ELISA, radioimmunoassay (RIA), fluorescent antibody, luminescence immunoassay (LIA), immunoprecipitation (IP), radial immunodiffusion (RID), turbidimetric immunoassay (TIA), western blot, immunohistochemical staining, and flow cytometry methods.

In various embodiments, a CEC of the present disclosure has a hexagonal morphology as detected by microscopy.

In various embodiments, a CEC of the present disclosure has a cytokine production profile of at least one of high PDGF-BB production, low IL-8 production, low MCP-1 production, high TNFα production, high IFNγ production, high IL-IR antagonist production, or low VEGF production. As used herein, “high production” and “low production” are relative and determined as being high or low compared to a generally observed level for each cytokine or the like. For instance, it is typically preferable that PDGF-BB is about 30 μg/ml or greater and IL-8 is about 500 μg/ml. Further, MCP-1 is preferably about 3000 μg/ml or less. TNFα is preferably about 10 μg/ml or greater, and IFNγ is preferably about 30 μg/ml or greater. IL-1R antagonists are preferably about 40 μg/ml or greater, and VEGF is preferably about 200-500 μg/ml or less.

In various embodiments, a CEC of the present disclosure does not have a karyotype abnormality (e.g., aneuploidy which may be induced in cell culture due to cell division). For humans, karyotype abnormalities can be measured in accordance with the Standard International System for Human Cytogenetic Nomenclature (ISCN) (1995) and definitions thereof.

In various embodiments, the CECs of the present disclosure are human (i.e., of human origin). In various embodiments, the CECs of the present disclosure are allogeneic. In various embodiments, the CECs of the present disclosure are cultured human CECs. In various embodiments, the CECs of the present disclosure are allogeneic cultured human CECs.

In various embodiments, the CECs of the present disclosure are sourced from a donor cornea. In various embodiments, the CECs are sourced from CECs of a donor cornea (e.g., are corneal endothelial tissue derived cells). In various embodiments, the CECs are not sourced from CECs of a donor cornea. In various embodiments, the CECs are differentiated from pluripotent stem cells (e.g., iPSCs), mesenchymal stem cells, corneal endothelial progenitor cells (e.g., precursor cells harvested from a corneal endothelium), cells harvested from a corneal endothelium, or corneal endothelial precursor cells or corneal endothelial-like cells produced via a direct programing method (e.g., corneal endothelial progenitor cells or corneal endothelial-like cells differentiated from stem cells, e.g., iPSCs). In various embodiments, the CECs are differentiated from pluripotent stem cells (e.g., iPSCs).

### CEC Compositions and Methods

In various embodiments, the disclosure provides compositions comprising CECs disclosed herein, wherein the compositions have improved shelf-life (e.g., improved shelf-life compared to CEC compositions in the art). In various embodiments, a composition of the present disclosure comprises a population of CECs (e.g., cultured human CECs) and a rho kinase (ROCK) inhibitor disclosed herein. In various embodiments, a composition of the present disclosure comprises a population of CECs (e.g., cultured human CECs) and a rho kinase (ROCK) inhibitor disclosed herein, and the composition has an improved shelf-life at a first temperature disclosed herein (e.g., when stored at a first temperature disclosed herein). In various embodiments, a composition disclosed herein having an improved shelf-life may be referred to as a stable composition (e.g., a stable CEC composition).

The present disclosure further provides methods related to the CEC compositions with improved shelf-life. In various embodiments, the present disclosure provides a method of improving shelf-life of a composition comprising CECs (e.g., cultured human CECs), the method comprising storing the composition at a first temperature disclosed herein, wherein the composition comprises a ROCK inhibitor disclosed herein. In various embodiments, the present disclosure provides a method of storing CECs (e.g., cultured human CECs), comprising storing the CECs with a ROCK inhibitor disclosed herein in a composition at a first temperature disclosed herein. In various embodiments, the present disclosure provides a method of preparing a CEC composition (e.g., a CEC composition comprising cultured human CECs), comprising storing the CEC composition at a first temperature disclosed herein, wherein the CEC composition comprises a ROCK inhibitor disclosed herein.

In various embodiments, the composition comprises about 1 μM to about 2000 μM, about 10 μM to about 2000 μM, about 50 μM to about 2000 μM, about 100 μM to about 2000 μM, about 250 μM to about 2000 μM, about 500 μM to about 2000 μM, about 750 μM to about 2000 μM, about 1000 μM to about 2000 μM, about 1250 μM to about 2000 μM, about 1500 μM to about 2000 μM, about 1750 μM to about 2000 μM, about 1 μM to about 1750 μM, about 10 μM to about 1750 μM, about 50 μM to about 1750 μM, about 100 μM to about 1750 μM, about 250 μM to about 1750 μM, about 500 μM to about 1750 μM, about 750 μM to about 1750 μM, about 1000 μM to about 1750 μM, about 1250 μM to about 1750 μM, about 1500 μM to about 1750 μM, about 1 μM to about 1500 μM, about 10 μM to about 1500 μM, about 50 μM to about 1500 μM, about 100 μM to about 1500 μM, about 250 μM to about 1500 μM, about 500 μM to about 1500 μM, about 750 μM to about 1500 μM, about 1000 μM to about 1500 μM, about 1250 μM to about 1500 μM, about 1 μM to about 1250 μM, about 10 μM to about 1250 μM, about 50 μM to about 1250 μM, about 100 μM to about 1250 μM, about 250 μM to about 1250 μM, about 500 μM to about 1250 μM, about 750 μM to about 1250 μM, about 1000 μM to about 1250 μM, about 1 μM to about 1000 μM, about 10 μM to about 1000 μM, about 50 μM to about 1000 μM, about 100 μM to about 1000 μM, about 250 μM to about 1000 μM, about 500 μM to about 1000 μM, about 750 μM to about 1000 μM, about 1 μM to about 750 μM, about 10 μM to about 750 μM, about 50 μM to about 750 μM, about 100 μM to about 750 μM, about 250 μM to about 750 μM, about 500 μM to about 750 μM, about 1 μM to about 500 μM, about 10 μM to about 500 μM, about 50 μM to about 500 μM, about 100 μM to about 500 μM, about 250 μM to about 500 μM, about 1 μM to about 250 μM, about 10 μM to about 250 μM, about 50 μM to about 250 μM, about 100 μM to about 250 μM, about 1 μM to about 100 μM, about 10 μM to about 100 μM, or about 50 μM to about 100 μM of the ROCK inhibitor. In various embodiments, the composition comprises about 10 μM to about 500 μM, about 50 μM to about 250 μM, about 80 μM to about 120 μM, or about 100 μM of the ROCK inhibitor. In various embodiments, the composition comprises about 100 μM of the ROCK inhibitor. In various embodiments, the composition comprises at least 1 μM, at least 10 UM, at least 20 μM, at least 30 μM, at least 40 μM, at least 50 μM, at least 60 μM, at least 70 μM, at least 80 μM, at least 90 μM, at least 100 μM, at least 150 μM, at least 200 μM, at least 500 μM, at least 1000 μM, at least 1250 μM, at least 1500 μM, at least 1750 μM, or at least 2000 μM of the ROCK inhibitor. In various embodiments, the composition comprises at least 1 μM, at least 10 UM, at least 20 μM, at least 30 μM, at least 40 μM, at least 50 μM, at least 60 μM, at least 70 μM, at least 80 μM, or at least 90 μM of the ROCK inhibitor.

In various embodiments, the ROCK inhibitor is 4-[(1R)-1-aminoethyl]-N-pyridin-4-cylcyclohexane-1-carboxamide. In various embodiments, the ROCK inhibitor is Y-27632. In various embodiments, the composition further comprises Dulbecco's modified eagle medium (DMEM). In various embodiments, the composition further comprises human serum albumin (HSA). In various embodiments, the composition further comprises human serum albumin (HSA) at 2%.

In various embodiments, a composition of the present disclosure has a shelf-life of at least 6 hours, at least 9 hours, at least 12 hours, at least 18 hours, at least 24 hours, at least 1.5 days, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 18 days, at least 21 days, at least 24 days, at least 27 days, at least 30 days, at least 40 days, at least 50 days, at least 60 days, at least 70 days, at least 80 days, at least 90 days, or at least 100 days at a first temperature disclosed herein (i.e., when stored at a first temperature disclosed herein). In various embodiments, a composition of the present disclosure comprising has a shelf-life of at least 6 hours, at least 9 hours, at least 12 hours, at least 18 hours, at least 24 hours, at least 1.5 days, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 18 days, at least 21 days, at least 24 days, at least 27 days, or at least 30 days at a first temperature disclosed herein (i.e., when stored at a first temperature disclosed herein). In various embodiments, a composition of the present disclosure has a shelf-life of at least 3 days, at least 5 days, at least 7 days, at least 9 days, or at least 10 days at a first temperature disclosed herein (i.e., when stored at a first temperature disclosed herein).

In various embodiments, a composition of the present disclosure has a shelf-life of about 3 days to about 7 days, about 3 days to about 8 days, about 3 days to about 9 days, about 3 days to about 10 days, about 3 days to about 11 days, about 3 days to about 12 days, about 3 days to about 13 days, about 3 days to about 14 days, about 3 days to about 15 days, about 3 days to about 18 days, about 3 days to about 21 days, about 3 days to about 24 days, about 3 days to about 27 days, about 3 days to about 30 days, about 3 days to about 40 days, about 3 days to about 50 days, about 3 days to about 60 days, about 3 days to about 70 days, about 3 days to about 80 days, about 3 days to about 90 days, about 3 days to about 100 days, about 5 days to about 7 days, about 5 days to about 8 days, about 5 days to about 9 days, about 5 days to about 10 days, about 5 days to about 11 days, about 5 days to about 12 days, about 5 days to about 13 days, about 5 days to about 14 days, about 5 days to about 15 days, about 5 days to about 18 days, about 5 days to about 21 days, about 5 days to about 24 days, about 5 days to about 27 days, about 5 days to about 30 days, about 5 days to about 40 days, about 5 days to about 50 days, about 5 days to about 60 days, about 5 days to about 70 days, about 5 days to about 80 days, about 5 days to about 90 days, about 5 days to about 100 days, about 7 days to about 9 days, about 7 days to about 10 days, about 7 days to about 11 days, about 7 days to about 12 days, about 7 days to about 13 days, about 7 days to about 14 days, about 7 days to about 15 days, about 7 days to about 18 days, about 7 days to about 21 days, about 7 days to about 24 days, about 7 days to about 27 days, about 7 days to about 30 days, about 7 days to about 40 days, about 7 days to about 50 days, about 7 days to about 60 days, about 7 days to about 70 days, about 7 days to about 80 days, about 7 days to about 90 days, or about 7 days to about 100 days at a first temperature disclosed herein (i.e., when stored at a first temperature disclosed herein). In various embodiments, a composition of the present disclosure has a shelf-life of about 7 days to about 9 days, about 7 days to about 10 days, about 7 days to about 11 days, about 7 days to about 12 days, about 7 days to about 13 days, about 7 days to about 14 days, about 7 days to about 15 days, about 7 days to about 16 days, about 7 days to about 17 days, about 7 days to about 18 days, about 7 days to about 19 days, or about 7 days to about 20 days at a first temperature disclosed herein (i.e., when stored at a first temperature disclosed herein).

In various embodiments, the first temperature is between about 10° C. and about 40° C., between about 10° C. and about 35° C., between about 10° C. and about 30° C., between about 10° C. and about 25° C., between about 10° C. and about 20° C., between about 10° C. and about 15° C., between about 15° C. and about 40° C., between about 15° C. and about 35° C., between about 15° C. and about 30° C., between about 15° C. and about 25° C., between about 15° C. and about 20° C., between about 20° C. and about 40° C., between about 20° C. and about 35° C., between about 20° C. and about 30° C., or between about 20° C. and about 25° C. In various embodiments, the first temperature is between about 10° C. and about 30° C., or between about 15° C. and about 25° C. In various embodiments, the first temperature is about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C. In various embodiments, the first temperature is about 15° C., about 16° C., about 17° C., about 18° C., about 19° C., about 20° C., about 21° C., about 22° C., about 23° C., about 24° C., or about 25° C. In various embodiments, the first temperature is at least 4° C., at least 5° C., at least 6° C., at least 7° C., at least 8° C., at least 9° C., or at least 10° C.

In various embodiments, a composition of the present disclosure has a shelf-life of at least 3 days at a temperature between about 10° C. and about 30° C., or between about 15° C. and about 25° C. In various embodiments, a composition of the present disclosure has a shelf-life of at least 3 days at (e.g., when stored at) about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C. In various embodiments, a composition of the present disclosure has a shelf-life of at least 3 days at (e.g., when stored at) about 15° C., about 16° C., about 17° C., about 18° C., about 19° C., about 20° C., about 21° C., about 22° C., about 23° C., about 24° C., or about 25° C.

In various embodiments, a composition of the present disclosure has a shelf-life of at least 5 days at a temperature between about 10° C. and about 30° C., or between about 15° C. and about 25° C. In various embodiments, a composition of the present disclosure has a shelf-life of at least 5 days at (e.g., when stored at) about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C. In various embodiments, a composition of the present disclosure has a shelf-life of at least 5 days at (e.g., when stored at) about 15° C., about 16° C., about 17° C., about 18° C., about 19° C., about 20° C., about 21° C., about 22° C., about 23° C., about 24° C., or about 25° C.

In various embodiments, a composition of the present disclosure has a shelf-life of at least 7 days at a temperature between about 10° C. and about 30° C., or between about 15° C. and about 25° C. In various embodiments, a composition of the present disclosure has a shelf-life of at least 7 days at (e.g., when stored at) about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C. In various embodiments, a composition of the present disclosure has a shelf-life of at least 7 days at (e.g., when stored at) about 15° C., about 16° C., about 17° C., about 18° C., about 19° C., about 20° C., about 21° C., about 22° C., about 23° C., about 24° C., or about 25° C.

In various embodiments, a composition of the present disclosure has a shelf-life of at least 9 days at a temperature between about 10° C. and about 30° C., or between about 15° C. and about 25° C. In various embodiments, a composition of the present disclosure has a shelf-life of at least 9 days at (e.g., when stored at) about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C. In various embodiments, a composition of the present disclosure has a shelf-life of at least 9 days at (e.g., when stored at) about 15° C., about 16° C., about 17° C., about 18° C., about 19° C., about 20° C., about 21° C., about 22° C., about 23° C., about 24° C., or about 25° C.

In various embodiments, a composition of the present disclosure has a shelf-life of at least 10 days at a temperature between about 10° C. and about 30° C., or between about 15° C. and about 25° C. In various embodiments, a composition of the present disclosure has a shelf-life of at least 10 days at (e.g., when stored at) about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C. In various embodiments, a composition of the present disclosure has a shelf-life of at least 10 days at (e.g., when stored at) about 15° C., about 16° C., about 17° C., about 18° C., about 19° C., about 20° C., about 21° C., about 22° C., about 23° C., about 24° C., or about 25° C.

In various embodiments, a composition of the present disclosure has a shelf-life of at least 15 days at a temperature between about 10° C. and about 30° C., or between about 15° C. and about 25° C. In various embodiments, a composition of the present disclosure has a shelf-life of at least 15 days at (e.g., when stored at) about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C. In various embodiments, a composition of the present disclosure has a shelf-life of at least 15 days at (e.g., when stored at) about 15° C., about 16° C., about 17° C., about 18° C., about 19° C., about 20° C., about 21° C., about 22° C., about 23° C., about 24° C., or about 25° C.

In various embodiments, a composition of the present disclosure has a shelf-life of at least 20 days at a temperature between about 10° C. and about 30° C., or between about 15° C. and about 25° C. In various embodiments, a composition of the present disclosure has a shelf-life of at least 20 days at (e.g., when stored at) about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., or about 30° C. In various embodiments, a composition of the present disclosure has a shelf-life of at least 20 days at (e.g., when stored at) about 15° C., about 16° C., about 17° C., about 18° C., about 19° C., about 20° C., about 21° C., about 22° C., about 23° C., about 24° C., or about 25° C.

In various embodiments, a composition of the present disclosure comprising CECs disclosed herein and a ROCK inhibitor disclosed herein has an improved shelf-life compared to a composition without a ROCK inhibitor. In various embodiments, a composition of the present disclosure comprising CECs disclosed herein and a ROCK inhibitor disclosed herein has an improved shelf-life compared to a composition not stored at a temperature disclosed herein (e.g., a first temperature disclosed herein).

As used herein, “shelf-life” refers to the length of time during which a composition of the present disclosure, when stored at a specified temperature (e.g., a temperature such as a first temperature disclosed herein), remains suitable for its intended use (e.g., use in treatment of a corneal endothelial disease). Unless expressly stated otherwise, shelf-life is determined from the time the composition is formulated (e.g., with a ROCK inhibitor) and maintained at the specified storage conditions, and is satisfied when, at the end of the stated period, the CECs retain desired characteristics disclosed herein (e.g., viability, cell adhesion, or phenotype) following gentle resuspension. For avoidance of doubt, a statement that a composition has a “shelf-life of at least about [X] days” means that, when stored continuously under the specified conditions for at least the stated number of days, the composition meets the foregoing criteria at the end of that period; longer durations may likewise constitute the shelf-life if the same criteria are met.

In various embodiments, the shelf-life of a composition of the present disclosure is represented by at least 50%, at least 55%, at least 60%, at least 65%, at least 70%, at least 75%, at least 80%, at least 85%, or at least 90% viability of the population of the CECs (e.g., the cultured human CECs). In various embodiments, the shelf-life of a composition of the present disclosure is represented by at least 70% viability of the population of the CECs (e.g., the cultured human CECs). In various embodiments, the shelf-life of a composition of the present disclosure is represented by at least 75% viability of the population of the CECs (e.g., the cultured human CECs). In various embodiments, the shelf-life of a composition of the present disclosure is represented by at least 80% viability of the population of the CECs (e.g., the cultured human CECs). In various embodiments, a method of the present disclosure comprises a step of identifying the viability of the CECs.

In various embodiments, the shelf-life of a composition of the present disclosure is represented by improved cell adhesion of the population of the CECs (e.g., the cultured human CECs). Cell adhesion may be mediated through transmembrane adhesion molecules linked to the intracellular cytoskeleton, and in various embodiments refers to the capability of the CECs (e.g., the cultured human CECs) to adhere to a substrate (e.g., to the Descemet's membrane when used in a therapy for a corneal endothelial dysfunction). In various embodiments, cell adhesion may be represented by adhesions of the CECs (e.g., the cultured human CECs) to a substrate, such as collagen of a collagen coated cell culturing dish, and quantified by the number of attached cells. In various embodiments, cell adhesion may be represented by expression or production of ZO-1, actin, and/or vinculin of the CECs (e.g., the cultured human CECs). In various embodiments, a population of CECs of a composition of the present disclosure comprising the population of CECs (e.g., a population of cultured human CECs) and a rho kinase (ROCK) inhibitor disclosed herein has a cell adhesion improvement of at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, at least 5-fold, at least 6-fold, at least 7-fold, at least 8-fold, at least 9-fold, or at least 10-fold. In various embodiments, a method of the present disclosure comprises a step of identifying the cell adhesion of the CECs.

Cell count, cell viability, and cell adhesion may be measured by methods known in the art, such as through flow cytometry, phase contrast images and actin fiber staining, CellTiter-Glo® luminescent cell viability assay (Promega), or an automated cell counter platform such as the NucleoCounter® NC-202™ counter using the Via2-Cassette™.

In various embodiments, a composition of the present disclosure comprising a population of CECs (e.g., a population of cultured human CECs) comprises about 100 μM of Y-27632, and at least 70% of the population of CECs (e.g., the population of cultured human CECs) are viable after storage for at least 3 days, at least 5 days, at least 7 days, or at least 10 days at a temperature between about 15° C. and about 25° C.

In various embodiments, a composition of the present disclosure comprising a population of CECs (e.g., a population of cultured human CECs) comprises about 100 μM of Y-27632, and at least 75% of the population of CECs (e.g., the population of cultured human CECs) are viable after storage for at least 3 days, at least 5 days, at least 7 days, or at least 10 days at a temperature between about 15° C. and about 25° C.

In various embodiments, a composition of the present disclosure comprising a population of CECs (e.g., a population of cultured human CECs) comprises about 100 μM of Y-27632, and at least 80% of the population of CECs (e.g., the population of cultured human CECs) are viable after storage for at least 3 days, at least 5 days, at least 7 days, or at least 10 days at a temperature between about 15° C. and about 25° C.

In some embodiments, provided herein are compositions comprising a population of at least 1×10⁵ corneal endothelial cells (CECs) and a rho kinase (ROCK) inhibitor, wherein at least 70% of the cells, e.g., at least 70%, 75%, or 80% of the cells, in the population of corneal endothelial cells are viable for 7 days when stored under appropriate conditions, e.g., 20 degrees Celsius. In certain embodiments, at least 70% of the cells, e.g., at least 70%, 75%, or 80% of the cells, in the population of CECs are viable for 10, 14, 18, 21, 24, 30, 40, 50, or 60 days when stored as indicated. In certain embodiments, at least 70% of the cells, e.g., at least 70%, 75%, or 80% of the cells, in the population of CECs are viable for 10, 14, 18, 21, 24, 30, 40, 50, or 60 days when stored under appropriate conditions. In certain embodiments, at least 70% of the cells, e.g., at least 70%, 75%, or 80% of the cells, in the population of CECs are viable for up to 21, 24, 30, 40, 50, or 60 days when stored under appropriate conditions. In certain embodiments, at least 70% of the cells, e.g., at least 70%, 75%, or 80% of the cells, in the population of CECs are viable for up to 90, 120, or 180 days when stored under appropriate conditions.

In various embodiments, in addition to the cellular component of the stable CEC composition, the present composition includes a ROCK inhibitor. The ROCK inhibitor can be prepared as a neutral or salt form or other prodrug (e.g., ester or the like). Pharmaceutically acceptable salts include salts formed with a free carboxyl group, derived from hydrochloric acid, phosphoric acid, acetic acid, oxalic acid, tartaric acid or the like, salts formed with a free amine group, derived from isopropylamine, triethylamine, 2-ethylaminoethanol, histidine, procaine or the like, and salts derived from sodium, potassium, ammonium, calcium, ferric hydroxide or the like.

In the present disclosure, “Rho kinase” or “ROCK” (Rho-associated coiled-coil forming kinase: Rho-bound kinase) refers to serine/threonine kinase which is activated with activation of Rho. Examples thereof include ROKalpha (ROCK-II: Leung, T. et al., J. Biol. Chem., 270, 29051-29054, 1995), p160ROCK (ROKbeta, ROCK-I: Ishizaki, T. et al., The EMBO J., 15 (8), 1885-1893, 1996) and other proteins having serine/threonine kinase activity.

Examples of ROCK inhibitors used as a combined agent include, without limitation, compounds disclosed in U.S. Pat. No. 4,678,783, Japanese Patent No. 3421217, WO 95/28387, WO 99/20620, WO 99/61403, WO 02/076976, WO 02/076977, WO 2002/083175, WO 02/100833, WO 03/059913, WO 03/062227, WO 2004/009555, WO 2004/022541, WO 2004/108724, WO 2005/003101, WO 2005/039564, WO 2005/034866, WO 2005/037197, WO 2005/037198, WO 2005/035501, WO 2005/035503, WO 2005/035506, WO 2005/080394, WO 2005/103050, WO 2006/057270, WO 2007/026664. Such compounds can be manufactured by the method described in each disclosed document. Examples thereof include 1-(5-isoquinolinesulfonyl) homopiperazine or a salt thereof (e.g., fasudil or fasudil hydrochloride), (+)-trans-4-(1-aminoethyl)-1-(4-pyridylcarbamoyl)cyclohexanecarboxamide or a salt thereof (e.g., Y-27632 ((R)-(+)-trans-(4-pyridyl)-4-(1-aminoethyl)-cyclohexanecarboxamide dihydrochloride monohydrate), and the like), and preferably comprising Y-27632. In various embodiments, the ROCK inhibitor is Y-27632.

In various embodiments, at least 50%, at least 55%, at least 60%, at least 65%, at least 70%, at least 75%, or at least 80% of the population of CECs (e.g., the population of cultured human CECs) of a composition disclosed herein have a phenotype disclosed herein (e.g., a cell surface marker phenotype disclosed herein). In various embodiments, a method of the present disclosure comprises a step of identifying the phenotype (e.g., a cell surface marker phenotype disclosed herein) of the CECs.

In various embodiments, at least 50%, at least 55%, at least 60%, at least 65%, at least 70%, at least 75%, or at least 80% of the population of CECs (e.g., the population of cultured human CECs) of a composition disclosed herein are CD166 positive. In various embodiments, at least 50%, at least 55%, at least 60%, at least 65%, at least 70%, at least 75%, or at least 80% of the population of CECs (e.g., the population of cultured human CECs) of a composition disclosed herein are CD166 positive and CD105 negative to weakly positive. In various embodiments, at least 50%, at least 55%, at least 60%, at least 65%, at least 70%, at least 75%, or at least 80% of the population of CECs (e.g., the population of cultured human CECs) of a composition disclosed herein are CD166 positive and CD105 negative. In various embodiments, at least 50%, at least 55%, at least 60%, at least 65%, at least 70%, at least 75%, or at least 80% of the population of CECs (e.g., the population of cultured human CECs) of a composition disclosed herein are CD166 positive, CD105 negative to weakly positive, and CD44 negative to weakly positive. In various embodiments, at least 50%, at least 55%, at least 60%, at least 65%, at least 70%, at least 75%, or at least 80% of the population of CECs (e.g., the population of cultured human CECs) of a composition disclosed herein are CD166 positive, CD105 negative, and CD44 negative. In various embodiments, at least 50%, at least 55%, at least 60%, at least 65%, at least 70%, at least 75%, or at least 80% of the population of CECs (e.g., the population of cultured human CECs) of a composition disclosed herein are CD24 negative. In various embodiments, at least 50%, at least 55%, at least 60%, at least 65%, at least 70%, at least 75%, or at least 80% of the population of CECs (e.g., the population of cultured human CECs) of a composition disclosed herein are CD26 negative. In various embodiments, at least 50%, at least 55%, at least 60%, at least 65%, at least 70%, at least 75%, or at least 80% of the population of CECs (e.g., the population of cultured human CECs) of a composition disclosed herein are CD166 positive, CD105 negative to weakly positive, CD24 negative, and CD44 negative to weakly positive. In various embodiments, at least 50%, at least 55%, at least 60%, at least 65%, at least 70%, at least 75%, or at least 80% of the population of CECs (e.g., the population of cultured human CECs) of a composition disclosed herein are CD166 positive, CD105 negative, CD24 negative, and CD44 negative. In various embodiments, a phenotype of CD166 positive, CD105 negative to weakly positive, CD24 negative, and CD44 negative to weakly positive, or a phenotype of CD166 positive, CD105 negative, CD24 negative, and CD44 negative, is referred to as E-ratio (effector ratio) in the present disclosure. In various embodiments, at least 50%, at least 55%, at least 60%, at least 65%, at least 70%, at least 75%, or at least 80% of the population of CECs (e.g., the population of cultured human CECs) of a composition disclosed herein are CD166 positive, CD105 negative to weakly positive, CD26 negative, and CD44 negative to weakly positive. In various embodiments, at least 50%, at least 55%, at least 60%, at least 65%, at least 70%, at least 75%, or at least 80% of the population of CECs (e.g., the population of cultured human CECs) of a composition disclosed herein are CD166 positive, CD105 negative, CD26 negative, and CD44 negative. In various embodiments, at least 50%, at least 55%, at least 60%, at least 65%, at least 70%, at least 75%, or at least 80% of the population of CECs (e.g., the population of cultured human CECs) of a composition disclosed herein are CD166 positive, CD105 negative to weakly positive, CD26 negative, CD24 negative, and CD44 negative to weakly positive. In various embodiments, at least 50%, at least 55%, at least 60%, at least 65%, at least 70%, at least 75%, or at least 80% of the population of CECs (e.g., the population of cultured human CECs) of a composition disclosed herein are CD166 positive, CD105 negative, CD26 negative, CD24 negative, and CD44 negative. In various embodiments, at least 50%, at least 55%, at least 60%, at least 65%, at least 70%, at least 75%, or at least 80% of the population of CECs (e.g., the population of cultured human CECs) of a composition disclosed herein are CD90 negative to weakly positive. In various embodiments, at least 50%, at least 55%, at least 60%, at least 65%, at least 70%, at least 75%, or at least 80% of the population of CECs (e.g., the population of cultured human CECs) of a composition disclosed herein are CD90 negative to weakly positive, CD133 negative, and CD26 negative. In various embodiments, at least 50%, at least 55%, at least 60%, at least 65%, at least 70%, at least 75%, or at least 80% of the population of CECs (e.g., the population of cultured human CECs) of a composition disclosed herein are CD90 negative to weakly positive, CD133 negative, and CD24 negative. In various embodiments, at least 50%, at least 55%, at least 60%, at least 65%, at least 70%, at least 75%, or at least 80% of the population of CECs (e.g., the population of cultured human CECs) of a composition disclosed herein are CD90 negative to weakly positive, CD133 negative, CD26 negative, and CD24 negative. In various embodiments, at least 50%, at least 55%, at least 60%, at least 65%, at least 70%, at least 75%, or at least 80% of the population of CECs (e.g., the population of cultured human CECs) of a composition disclosed herein are CD90 negative to weakly positive, CD133 negative, CD166 positive, CD105 negative, CD24 negative, and CD44 negative. In various embodiments, at least 50%, at least 55%, at least 60%, at least 65%, at least 70%, at least 75%, or at least 80% of the population of CECs (e.g., the population of cultured human CECs) of a composition disclosed herein are CD90 negative to weakly positive, CD133 negative, CD166 positive, CD105 negative, CD26 negative, and CD44 negative.

In various embodiments, the population of CECs (e.g., the population of cultured human CECs) of a composition disclosed herein has one or more of the following characteristics:
(1) when assessed by culture supernatant enzyme-linked immunoassay assay (ELISA):
   TIMP-1:500 ng/mL or less
   IL-8:500 μg/mL or less
   PDGF-BB: 30 μg/mL or greater
   MCP-1:3000 μg/mL or less
(2) when assessed by fluorescence-activated cell sorting (FACS):
   CD166 positive=95% or greater
   CD133 positive=5% or less
   CD105 low positive=95% or greater
   CD44 low positive=80% or greater
   CD44 high positive=5% or less
   CD24 positive=5% or less
   CD26 positive=5% or less
   CD200 positive=5% or less
(3) barrier function (ZO-1) positive
(4) pumping function (Na+/K+ ATPase) positive
(5) CD44 strongly positive cell <5%, CD26 positive cell <5%, or CD24 positive cell <5%

(6) karyotype abnormality negative.

In various embodiments, the population of CECs (e.g., the population of cultured human CECs) of a composition disclosed herein are allogeneic. In various embodiments, the population of CECs of a composition disclosed herein are allogeneic cultured human CECs.

In various embodiments, the population of CECs (e.g., the population of cultured human CECs) of a composition disclosed herein are sourced from a donor cornea. In various embodiments, the population of CECs are sourced from CECs of a donor cornea (e.g., are corneal endothelial tissue derived cells). In various embodiments, the population of CECs are not sourced from CECs of a donor cornea. In various embodiments, the population of CECs are differentiated from pluripotent stem cells (e.g., iPSCs), mesenchymal stem cells, corneal endothelial progenitor cells (e.g., precursor cells harvested from a corneal endothelium), cells harvested from a corneal endothelium, or corneal endothelial precursor cells or corneal endothelial-like cells produced via a direct programing method (e.g., corneal endothelial progenitor cells or corneal endothelial-like cells differentiated from stem cells, e.g., iPSCs). In various embodiments, the population of CECs are differentiated from pluripotent stem cells (e.g., iPSCs).

In various embodiments, a composition of the present disclosure comprises at least about 1×10⁵, at least about 2.5×10⁵, at least about 5×10⁵, at least about 7.5×10⁵, at least about 1×10⁶, at least about 2.5×10⁶, at least about 5×10⁶, at least about 7.5×10⁶ CECs (e.g., cultured human CECs). In various embodiments, a composition of the present disclosure comprises at least about 1×10⁶ CECs (e.g., cultured human CECs).

In various embodiments, the population of CECs (e.g., the population of cultured human CECs) of a composition disclosed herein achieves a mean cell density of at least about 1500 cells/mm² or higher, at least about 1600 cells/mm² or higher, at least about 1700 cells/mm² or higher, at least about 1800 cells/mm² or higher, at least about 1900 cells/mm² or higher, or at least about 2000 cells/mm² or higher after administration to a human subject (e.g., to an eye of the subject) and integration into a human corneal endothelial surface.

In various embodiments, the population of CECs (e.g., the population of cultured human CECs) is in a suspension form. As use herein, “suspension” refers to a non-adherent, re-suspendable preparation of cells in a liquid vehicle in which the cells remain capable of being maintained and/or redistributed throughout the liquid phase with minimal agitation. A “suspension” includes preparations in which the cells are present as single cells and/or as cell clusters, microaggregates, or larger aggregates, and encompasses states in which the cells are momentarily dispersed throughout the liquid or have settled or precipitated under gravity during storage but can be readily redispersed by gentle mixing. Without limitation, a “suspension” thus includes: (i) cells uniformly dispersed in the medium; (ii) cells that have partially or fully settled to the bottom of the container over time but are not substantially adherent and can be redispersed by inversion, gentle shaking, or pipetting; and (iii) cells present as single cells, loose clusters, or compact aggregates that remain non-adherent to the container surface and maintain the capacity to be redistributed throughout the medium. A preparation is not a “suspension” if the cells are substantially adherent to the container surface such that they cannot be redistributed throughout the liquid phase without enzymatic treatment or forceful mechanical detachment. In various embodiments, the population of CECs (e.g., the population of cultured human CECs) is not substantially adherent to a surface of a container (e.g., a vial) of a composition comprising the population of CECs. In various embodiments, “substantially adherent to a surface of a container” refers to at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population of CECs adhering to the surface of the container. In various embodiments, the population of CECs (e.g., the population of cultured human CECs) is in a suspension form or is not substantially adherent to a surface of a container (e.g., a vial) of a composition comprising the population of CECs. In various embodiments, the population of CECs (e.g., the population of cultured human CECs) is in a suspension form or is not substantially adherent to a surface of a container (e.g., a vial) of a composition comprising the population of CECs, while the population of CECs maintains a cell adhesion characteristic disclosed herein (e.g., capability of a CEC to adhere to a substrate).

The present disclosure therefore also provides pharmaceutical formulations and storage conditions for cell compositions (e.g., comprising a CEC disclosed herein or a suitable cell) for enhanced shelf-life, and methods of formulating and storing cell compositions (e.g., comprising a CEC disclosed herein or a suitable cell) for enhanced shelf-life. In various embodiments, a pharmaceutical formulation that enhances shelf-life of a cell composition comprises Dulbecco's Modified Eagle's Medium (DMEM) and a Rho-associated protein kinase (ROCK)-inhibitor. In various embodiments, a pharmaceutical formulation that enhances shelf-life of a cell composition comprises Dulbecco's Modified Eagle's Medium (DMEM), Human Serum Albumin (HSA), and a Rho-associated protein kinase (ROCK)-inhibitor (e.g., at an amount disclosed herein). In various embodiments, the pharmaceutical formulation comprises 2% HSA. In various embodiments, the ROCK inhibitor is Y-27632. In various embodiments, the formulation comprises DMEM, 2% HSA, and Y-27632 (e.g., about 100 μM Y-27632, or an amount disclosed herein).

In various embodiments, the pharmaceutical formulation preserves greater than about 50%, about 55%, about 60%, about 65%, about 70%, about 75%, about 80%, about 85%, about 90%, or about 95% viability of cells of the cell composition formulated with the pharmaceutical formulation, when the formulated composition is stored at a temperature disclosed herein (e.g., between about 10° C. and about 30° C., or between about 15° C. and about 25° C.). In various embodiments, the pharmaceutical formulation preserves cell adhesion. In various embodiments, a cell composition formulated with the pharmaceutical formulation has improved cell adhesion by at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, at least 5-fold, at least 6-fold, at least 7-fold, at least 8-fold, at least 9-fold, or at least 10-fold, in cells of the cell composition formulated with the pharmaceutical formulation, when the formulated composition is stored at a temperature disclosed herein (e.g., between about 10° C. and about 30° C., or between about 15° C. and about 25° C.), as compared to cells not formulated with the pharmaceutical formulation.

### Methods of Culturing Corneal Endothelial Cells (CECs)

A cornea is one of the lamellar tissues constituting an eye. In humans, the cornea is composed of five layers, corneal epithelium, Bowman's membrane (external boundary), Lamina propria, Descemet's membrane (internal boundary), and corneal endothelium, in order from the outside (body surface) to the inside. The corneal endothelium is a single layer of cells that covers the posterior cornea. Markers for characterizing CECs and methods of identifying CECs are known in the art (See e.g., Hamuro J, et al. Invest Ophthalmol Vis Sci. 2016 Aug. 1; 57 (10): 4385-92; Wongvisavavit, R., et al (2021). Regenerative medicine, 16 (09), 871-891).

Robust proliferation, expansion, and the ability to store the cultured human CECs as a stable cell population is important for CEC therapy. The compositions and methods disclosed herein provide a stable CEC populations that can be used in CEC therapy to treat corneal endothelial diseases.

In some embodiments, provided herein are stable cell compositions comprising a population of human CECs and a ROCK inhibitor. The population of CECs comprises at least 1×10⁵ CECs at the time of harvest, wherein the composition has at least 70% cell viability following seven days of storage under appropriate conditions, e.g., 20 degrees Celsius. Other desirable properties of the cell population are provided infra.

The CECs, CEC compositions, and stable cell populations provided herein are not limited by the culturing method used to obtain the cells of the CECs, CEC compositions, and cell populations. However, exemplary culturing methods that increase the number of CECs (e.g., human corneal endothelial cells (CECs)) that are expanded from a single cornea are provided herein. The exemplary method includes culturing CECs (e.g., human CECs) in an initial culture (Passage 0; P0) followed by at least four additional passages (e.g., passage 1 (P1), passage 2 (P2), passage 3 (P3), and passage 4 (P4)). In certain embodiments, the method provides enough CECs that a thousand or more doses of CECs can be used for CEC-based therapies.

For example, a method of culturing CECs (e.g., human corneal endothelial cells (CECs)) that can be used, includes culturing CECs (e.g., human corneal endothelial cells (CECs)) in an initial passage 0 (P0); and expanding the human CECs through at least four additional passages comprising passage 1 (P1), passage 2 (P2), passage 3 (P3), and passage 4 (P4), wherein both P3 and P4 have time periods that are shorter than each of P0, P1, and P2.

Cells from later passages can be used, including expanding the CECs through passage 5 (P5). In some embodiments, P5 is shorter in duration than each of P0, P1, and P2.

In a further embodiment, the methods include culturing the CECs at P0 for at least 35 days, e.g., for 35 to 80 days.

In another embodiment, the methods include expanding the CECs at P1 for at least 30 days, e.g., for 30-80 days.

In one embodiment, the methods include expanding the CECs at P2 for at least 30 days, e.g., for 30-80 days.

In still other embodiments, the methods include expanding the CECs at P3 for at least 20 days, e.g., for 20 to 45 days or for 20 to 30 days.

In a further embodiment, the methods include expanding the CECs for at least 20 days, e.g., for 20 to 30 days.

An alternative example of a method of culturing corneal endothelial cells includes culturing corneal endothelial cells (CECs) at passage 0 (P0) for at least 30 days; expanding the CECs at passage 1 (P1) for at least 30 days; expanding the CECs at passage 2 (P2) for at least 30 days; expanding the CECs at passage 3 (P3) for less than 30 days; and expanding the CECs at passage 4 (P4) for less than 30 days. Optionally, the method further includes expanding the CECs at passage 5 (P5) for less than 30 days.

To provide cells for a larger number of patients, the stable cell compositions and methods provided herein typically include the use of cultured cells from P3 or later. However, the stable cell compositions and methods herein are dependent on the quality of the CECs, e.g., viability and characteristic CEC properties, more than the particular passage number. Such considerations are understood by those of skill in the art.

Methods for CEC culture can include methods wherein each of the passages is performed at the same temperature. For example, each passage can be performed at a temperature of at least about 31° C., e.g., at a temperature of about 37° C. or at a temperature of about 31° C. to 41° C.

Methods for CEC culture can include methods wherein the CECs are derived from a single donor.

Methods for CEC culture can include methods wherein any one of P0, P1, P2, P3, or P4 are performed in a cell culture medium supplemented with ascorbic acid, fetal bovine serum, chondroitin sulfate, calcium chloride, or a Rho kinase inhibitor. For example, the Rho kinase inhibitor is Y-27632.

CECs cultured by methods disclosed herein have advantageous properties useful for the compositions and methods provided here. For example, at least 70% of the CECs are viable at the end of P4 as determined by a cell viability assay. In some populations, at least 90% of the CECs are viable at the end of P4 as determined by a cell viability assay.

In various embodiments, cells cultured by methods disclosed herein have a phenotype disclosed herein. In various embodiments, cells cultured by methods disclosed herein have at least 70% of the CECs having a cell surface expression at the end of P4 of a marker selected from the group consisting of CD166 positive, CD44 negative to CD44 weakly positive, CD24 negative to weakly positive, CD44 negative to weakly positive, CD105 negative to weakly positive, CD26 negative to weakly positive, CD200 negative to weakly positive, and CD90 negative to weakly positive phenotypes.

In some embodiments, at least 70% of the CECs have a cell surface expression at the end of P4 of a marker selected from the group consisting of sodium-potassium ATPase, ZO-1, VDAC3, SLC4A4, CLCN3, COL4A2, COL8A1, COL8A2, CDH2, CD98, CD166, CD340, Integrin α3β1, CD56, Prdx-6, CD248, SLC4A11, and CYYR1.

In some embodiments, at least 70% of the CECs at the end of P4 have a hexagonal morphology as detected by microscopy.

In some embodiments, the cells are optionally grown on a cell culture surface comprising an extracellular matrix or an extracellular matrix protein. Extracellular matrix proteins include, for example, collagen, laminin, fibronectin, proteoglycan, SWI/SNF-related matrix-associated actin-dependent regulator of chromatin subfamily A, SWI/SNF-related matrix-associated actin-dependent regulator of chromatin subfamily B member 1, insulin-like growth factor-binding protein 5, vitronectin, fibrillin-1, fibrilin-2, tensin, Wnt-5b, citron Rho-interacting kinase, chondroitin sulphate proteoglycan 4, cyclin-dependent kinase 1, cyclin-dependent kinase 4, periostin, thrombospondin-4, Tubulin alpha chain-like 3, Tubulin alpha-1B chain, Tubulin beta-1 chain, Tubulin beta-4A chain, or versican core protein.

The culture vessel is optionally tissue-culture (TC)-treated.

### Collection of Corneal Endothelial Cells and Culture Thereof in a Culture Vessel In Vitro

Corneal endothelial cells (e.g., human CECs) may be collected by any conventional methods known in the art from the cornea of a suitable corneal donor. In brief, the CECs may be isolated by stripping Descemet's membrane, followed by enzyme treatment to remove the collagen matrix. These cells may undergo further analysis to confirm their biological characteristics and to verify criteria for therapeutic use. Markers for characterizing CECs and methods of identifying CECs are known in the art (See, e.g., Hamuro J, Ueno M, Toda M, Sotozono C, Montoya M, Kinoshita S. Invest Ophthalmol Vis Sci. 2016 Aug. 1; 57 (10): 4385-92; and Wongvisavavit, R., et al (2021). Regenerative medicine, 16 (09), 871-891).

In some embodiments, the CECs are from a human CEC primary cell line. Homogeneous corneal endothelial cells may be prepared using methods known in the art. For example, the Descemet's membrane and the endothelial cell layer of a corneal tissue may be detached from the corneal stroma, transferred into a culture vessel (e.g., a culture dish), and treated with an enzyme, such as collagenase A. In some embodiments, the CECs with Descemet's membrane and the endothelial cell layer are digested in a basal growth medium (e.g., OPTI-MEM® I Reduced Serum Media (Thermo Fisher Scientific, Inc., e.g., free of ammonium meta vanadate combined with fetal bovine serum (e.g., 8%)), which may be supplemented with additional components such as calcium chloride (e.g., 200 mg/L), chondroitin sulfate (e.g., 0.08%), and/or an antibiotic (e.g., gentamicin). The CECs may be digested at 37° C. for two to 24 hours. As a result, the corneal endothelial cells are detached from the Descemet's membrane. The corneal endothelial cells remaining in the Descemet's membrane can be further detached by mechanical methods, such as pipetting. This step may additionally include one or more washing steps (e.g., using the basal growth medium without an enzyme).

After removal of the Descemet's membrane, the corneal endothelial cells may then be cultivated in a suitable culture medium that permits growth of CECs (e.g., in an initial culture at passage 0). For example, in some embodiments, the CECs are cultured in a basal growth medium (e.g., OPTI-MEM® I Reduced Serum Media (Thermo Fisher Scientific, Inc., e.g., free of ammonium meta vanadate and combined with fetal bovine serum (e.g., 8%)). In some embodiments, the basal growth medium is further supplemented with an epidermal growth factor (EGF) and/or ascorbic acid (e.g., 20 μg/mL). In one embodiment, the basal growth medium further comprises a Rho-associated protein kinase (ROCK)-inhibitor, such as Y-27632. As a further example, commercially available DMEM (Dulbecco's Modified Eagle's Medium) appropriately supplemented with FBS (fetal bovine serum), b-FGF (basic-fibroblast growth factor) and antibiotics such as penicillin, streptomycin and the like can be used.

In two-dimensional culture systems, adherent cells are grown in a monolayer system on a flat surface, e.g. in a culture dish, plate, or flask. In some embodiments, the culture vessel has a surface coated with Type I collagen, Type IV collagen, fibronectin, laminin or an extracellular matrix of bovine corneal endothelial cells and the like. Alternatively, a conventional culture vessel treated with a commercially available coating agent, such as an FNC coating mix may be used.

The temperature for cultivating corneal endothelial cells is not limited as long as the cells proliferate. For example, CECs can be cultured at a temperature of about 31° C. to about 41° C. In certain embodiments, the cells are cultured at a temperature of about 37° C. The cells may be cultured at a temperature of about 32° C. The cells may be cultured at a temperature of about 41° C. The cells may be incubated in a conventional incubator for cell culture under humidification in an environment of about 5-10% CO₂.

### Passaging

Following the initial culture (passage 0), the cultured corneal endothelial cells (CECs) are passaged into fresh growth medium. Subculturing cells increases the number of cells that may be expanded from a single donor (e.g., a human corneal donor).

The passage or subculture may include one or more of the following steps. First, spent media may be removed and the cells may be detached from the surface of the culture vessel (e.g., by a treatment with mechanical disruption or an agent, e.g., trypsin-EDTA, TrypLE enzyme, by scraping, by shaking, etc.) and recovered. At the time the cells are detached, the cells may be sub-confluent or confluent. A culture medium may then be added to the recovered cells to generate a cell suspension. In some embodiments, centrifugation may be performed during or after recovery of the cells (e.g., 500 rpm (30 G)-1000 rpm (70 G), 1 to 10 minutes). Such centrifugal treatment generates a cell suspension with a high cell density.

The cell suspension may then be seeded and cultured in a culture vessel. The dilution ratio during passage may vary depending on the condition of the cells. In some embodiments, the dilution ratio during passage is about 1:2-1:4. In some embodiments, the dilution ratio is about 1:3. The culture time may vary depending on the condition of the cells to be used.

The passages after the initial culture (P0) are alternatively referred to herein as Passage 1 (P1), Passage 2 (P2), Passage (P3), Passage (P4), and so on. In some embodiments, the cultured CECs are passaged at least four times (e.g., prior to collecting the CECs for incorporation into a stable CEC composition or a CEC composition disclosed herein). In certain embodiments, the cultured CECs are passaged at least five times (e.g., prior to collecting the CECs for incorporation into a stable CEC composition or a CEC composition disclosed herein). It is understood that culture methods can include initially culturing the cell (P0) and expanding the cells via more than five additional passages (e.g., P1, P2, P3, P4, P5, P6, or more) prior to collecting the cells.

In some embodiments, the method includes expanding the CECs through one or more additional passages beyond P5 (e.g., P6, P7, and so forth).

The temperature for cultivating corneal endothelial cells is not limited as long as the cells proliferate. For example, in some embodiments, the cells are passaged at a temperature of about 31° C. to about 41° C. (e.g., about 32° C. to about 41° C., about 35° C. to about 41° C.). In certain embodiments, the cells are passaged at a temperature of at least about 31° C. (e.g., about 31° C., 32° C., 33° C., 34° C., 35° C., 36° C., 37° C., 38° C., 39° C., 40° C., 41° C.). In certain embodiments, the cells are passaged at a temperature of about 37° C. In certain embodiments, the cells are passaged at a temperature of about 32° C. In certain embodiments, the cells are passaged at a temperature of about 41° C. The cells may be incubated in a conventional incubator for cell culture under humidification in an environment of about 5-10% CO₂.

In some embodiments, each of the passages are performed at the same temperature. In certain embodiments, each passage (i.e., P0, P1, P2, P3, P4, and optionally, P5 or later passages) is performed at about 37° C. In some embodiments, one or more of the passages is performed at a different temperature relative to the other passages.

The CECs may be cultured in a variety of cell culture vessels. Clinical studies involving CECs have generally used polystyrene T-flasks for expanding the CECs. However, the present method can alternatively be performed in other suitable culture vessels for later passages, such as layered or stacked culture chambers (e.g., Polystyrene CellSTACK™ chambers. Accordingly, in some embodiments, the cells are expanded in commercially available T-flasks (e.g., T-25, T-75, T-150, T-225) for P1, P2, P3, P4, and/or P5 (or passages beyond P5). In some embodiments, the CECs in P3 and/or P4 are expanded in a stacked cell culture chamber that includes two or more layers (e.g., 2-10 layers). Cell culture vessels having different volumes or surface areas may be utilized. For example, in some embodiments, the cell culture chamber has a medium volume of 130 to 8000 mL. In some embodiments, the cell culture chamber has a cell culture area of at least about 25 cm² to at least about 200 cm². In some embodiments, the cell culture chamber has about 600 to about 6400 cm² culture area. Further, different types or volumes of cell culture vessels can be used during the passaging process. In some embodiments, the volume and/or surface area of the cell culture chamber may be increased with each passage.

Cells for use in the stable cell compositions and methods provided herein can be grown in a cell culture vessel coated with a cell substrate. Examples of the aforementioned substrate include polymer materials derived from naturally-occurring substances such as collagen, gelatin, cellulose and the like, synthesized polymer materials such as polystyrene, polyester, polycarbonate, poly(N-isopropylacrylamide) and the like, biodegradable polymer materials such as polylactic acid, polyglycolic acid and the like, hydroxyapatite, amniotic membrane and the like. In one embodiment, the cell substrate is collagen (e.g., a collagen-coated plate).

CECs may be cultured in culture vessels coated with an extracellular matrix, or proteins therefrom, to promote cellular proliferation (see, e.g., San Choi, Jin, et al. Biomedical materials 8.1 (2013): 014108.; Okumura, Naoki, et al. Investigative Ophthalmology & Visual Science 56.5 (2015): 2933-2942.; Parekh, Mohit, et al. Acta Ophthalmologica 99.4 (2021): e512-e522; Blake, Diane A., et al. Investigative Ophthalmology & Visual Science 38.6 (1997): 1119-1129, which are hereby incorporated by reference). Accordingly, in some embodiments, the cells are grown on a cell culture surface comprising an extracellular matrix. For example, the extracellular matrix may be one derived from CECs (e.g., human CECs or bovine CECs). See, e.g., Blake et al (1997) and Parekh et al (2021).

Cells for use in the stable cell compositions and methods provided herein can be grown on a cell culture surface comprising an extracellular matrix protein. Exemplary extracellular matrix proteins include collagen (e.g., collagen type I, collagen type IV), laminin, fibronectin, proteoglycan, SWI/SNF-related matrix-associated actin-dependent regulator of chromatin subfamily A, SWI/SNF-related matrix-associated actin-dependent regulator of chromatin subfamily B member 1, insulin-like growth factor-binding protein 5, vitronectin, fibrillin-1, fibrilin-2, tensin, Wnt-5b, citron Rho-interacting kinase, chondroitin sulphate proteoglycan 4, cyclin-dependent kinase 1, cyclin-dependent kinase 4, periostin, thrombospondin-4, Tubulin alpha chain-like 3, Tubulin alpha-1B chain, Tubulin beta-1 chain, Tubulin beta-4A chain, and versican core protein. In certain embodiments, the extracellular matrix protein is collagen, laminin, or fibronectin. In one embodiment, the extracellular matrix protein is collagen.

Cells for use in the stable cell compositions and methods provided herein can be grown in a cell culture vessel that is a tissue-culture treated (TC-treated) cell culture vessel.

The CECs may be cultured and expanded in a suitable culture medium that permits growth of CECs. For example, the CECs are cultured in a basal growth medium (e.g., OPTI-MEM® I Reduced Serum Media (Thermo Fisher Scientific, Inc., e.g., free of ammonium meta vanadate and combined with fetal bovine serum (e.g., 8%)). In some culture methods, the basal growth medium is further supplemented with an epidermal growth factor (EGF) or ascorbic acid (e.g., 20 μg/mL). In some culture methods, the basal growth medium further comprises a Rho-associated protein kinase (ROCK)-inhibitor, such as Y-27632. In certain culture methods, any one of P0, P1, P2, P3, or P4 are performed in a cell culture medium supplemented with ascorbic acid, fetal bovine serum, chondroitin sulfate, calcium chloride, or a Rho kinase inhibitor. In certain culture methods, each of P0, P1, P2, P3, and P4 are performed in a cell culture medium supplemented with ascorbic acid, fetal bovine serum, chondroitin sulfate, calcium chloride, or a Rho kinase inhibitor. An exemplary cell culture media is supplemented with ascorbic acid, fetal bovine serum, chondroitin sulfate, calcium chloride, and a Rho kinase inhibitor.

After harvesting the cells at the desired passage, the cells can be formulated in a suitable medium, such as commercially available DMEM (Dulbecco's Modified Eagle's Medium). Optionally, the DMEM is supplemented with Human Serum Albumin (e.g., 2% HSA) and a Rho-associated protein kinase (ROCK)-inhibitor (e.g., Y-27632). Preferably, the DMEM is supplemented with a ROCK inhibitor (e.g., Y-27632).

The provided stable cell compositions comprise viable CECs that exhibit functional properties similar to CECs native to the corneal endothelium. Markers for characterizing or identifying CECs are known in the art (See e.g., Hamuro J, Ueno M, Toda M, Sotozono C, Montoya M, Kinoshita S. Invest Ophthalmol Vis Sci. 2016 Aug. 1; 57 (10): 4385-92; and Wongvisavavit, R., et al (2021). Regenerative medicine, 16 (09), 871-891). Any methods known in the art to assess cell markers can be used to characterize the CECs, such as flow cytometry, western blot, immunocytochemistry, immunohistochemistry, immunoprecipitation, quantitative PCR; and reverse transcription PCR. CECs additionally have a hexagonal morphology that is observable under a microscope.

The exemplary method provided results in a large number of viable CECs for use in stable compositions, including stable therapeutic compositions provided herein. Cells cultured by exemplary methods described herein have properties that make them useful in the stable compositions and methods provided herein including high viability, appropriate cell surface marker expression, and hexagonal morphology.

The resulting cells can be used to formulate CECs into stable CEC compositions of the invention, and are suitable for injection into an eye of a subject for the treatment of a corneal endothelial disease, as further described herein.

Provided herein are stable CEC compositions including corneal endothelial cells (e.g., for use in treating corneal endothelial disease). The CECs may be cultured, expanded, and harvested in accordance with the exemplary methods provided herein or any other appropriate method in the art to produce a stable CEC composition having the desired characteristics provided herein. In certain embodiments, the stable CEC composition can be administered intraocularly, e.g., intracamerally, for use as a therapeutic to treat corneal endothelial diseases.

Preferably, the compositions described herein are pharmaceutical compositions.

In some embodiments, the compositions herein include a stable CEC composition capable of cell proliferation in vivo, wherein the CEC were expanded in culture in vitro on a substrate in a cultured CEC layer. In some embodiments, CECs that are used to prepare the stable CEC compositions have been (1) cultured at least in a culture vessel (e.g., culture dish, culture tube, culture tank etc.), (2) such cells passage-cultured further (e.g., 3-10 passages), or (3) such passage-cultured cells that are further cultured on a substrate.

In some embodiments, the cultured corneal endothelial cell layer used to prepare the composition herein may have at least one, at least two, or all of the following characteristics prior to harvesting the cells prior formulation into the stable CEC composition. (1) The cell layer may have a monolayer structure. This is one of the characteristics of the corneal endothelial cell layer of living organisms. (2) The cell density of the cell layer may be about 1,000-about 4,000 cells/mm². Particularly, when the recipient (transplantee) is an adult, the density may be about 2,000-about 3,000 cells/mm². (3) The visual flat plane shape of the cell constituting the cell layer may be approximately hexagonal. This is one of the characteristics of the cell constituting the corneal endothelial cell layer in living organisms. (4) In the cell layer, cells are regularly aligned. In the corneal endothelial cell layer in living organisms, the cells constituting the layer are regularly aligned, by which it is considered that the corneal endothelial cells maintain normal function and high transparency and the cornea appropriately controls the water content. Having such morphological characteristics (e.g., hexagonal shape), the composition herein may have functions similar to those of the corneal endothelial cell layer in living organisms.

Further provided are pharmaceutical compositions including the stable CEC compositions described herein and a pharmaceutically acceptable carrier or excipient. As used herein, “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained. Such a composition contains a therapeutically effective amount of CECs together with a suitable amount of carrier, such that the composition is provided in a form suitable for administration to a patient, e.g., suitable for intraocular administration to a patient.

In certain embodiments, the stable CEC compositions are provided in appropriate packaging. For example, the stable CEC composition can be provided in a container or a vial. In certain embodiments, the container or the vial is appropriate for storage of a pharmaceutical composition for intracameral administration. In certain embodiments, the container or the vial is appropriate for storage of the population of cells, e.g., the cells are not substantially adherent to the surface of the container or the vial so that any cells can be released from the surface of the container or the vial using methods that would not damage the cells, e.g., gentle agitation or tapping, so that the appropriate dose of cells may be delivered to the patient.

In certain embodiments, the stable CEC composition is provided in a unit dosage form. A unit dosage form typically includes a sufficient volume to provide a single dose or to treat one person (e.g., where the person has two eyes requiring treatment). A unit dosage form includes a sufficient amount of the therapeutic with sufficient overage to allow the appropriate dose or doses to be withdrawn from the container or the vial and administered. In certain embodiments, the population of cells is 1.70×10⁶ cells to 5×10⁶ cells. In certain embodiments, the container or the vial contains at least 1.70×10⁶ CECs, at least 2×10⁶ CECs, at least 3×10⁶ CECs, or at least at least 5×10⁶ CECs or more. In certain embodiments, the unit dosage form contains a sufficient number of cells to permit dosing of about 1×10⁶ viable cells per eye.

In certain embodiments, the stable CEC composition, e.g., in a container or vial or a unit dosage form may be included in a kit, optionally further including label for use. In certain embodiments, the kit further includes a device for delivery. A kit does not require that all of the components be packaged in a single container, particularly if the components of the kit are stored under different conditions, e.g., refrigeration and room temperature.

### Methods of Administration and Treatment Using Stable CEC Compositions

In various embodiments, the present disclosure further provides methods and uses of CECs or CEC compositions (e.g., cultured human CEC compositions) disclosed herein. Further provided herein are methods of administration, e.g., intracameral administration, of a stable CEC composition or a CEC composition disclosed herein and methods of treating a corneal endothelial disease in a subject in need thereof by administering an effective amount of a stable corneal endothelial cell composition or a CEC composition provided herein to an eye of the subject. The CECs may be initially cultured, expanded, and harvested in accordance with the disclosed methods and combined with a ROCK inhibitor, e.g., Y-27632, to provide a stable CEC composition. In certain embodiments, the stable CEC composition is administered to a subject at least 7 days after the cells were harvested. In certain embodiments, the stable CEC composition is administered to a subject at least 10, 14, 18, 21, 25, or 30 days after the cells were harvested.

In certain embodiments, the CEC composition is acceptable for administration to a subject, e.g., intracameral delivery to a subject, for treatment of corneal endothelial disease at 7 days after harvest. In certain embodiments, the CEC composition is acceptable for administration to a subject for treatment 10, 14, 18, 21, 25, 30, or 60 days after harvest. In certain embodiments, the CEC composition is acceptable for administration to a subject for up to 21, 24, 30, 40, 50, or 60 days after harvest. In certain embodiments, the CEC composition is acceptable for administration to a subject for up to 90, 120, or 180 days after harvest. Acceptable for administration can be understood as in a population of at least 1×10⁶ viable cells, the viability of the cell population is at least 70%, the immunophenotyping E-ratio is at least 75% (i.e., at least 75% of the cell population expresses the phenotype of E-ratio), and the adhesion as assayed by attached cells/well is at least 8.5×10⁴ cells/well.

In various embodiments, a composition of the present disclosure comprising a population of CECs (e.g., a population of cultured human CECs) and a rho kinase (ROCK) inhibitor disclosed herein, before administration, is stored for at least 6 hours, at least 9 hours, at least 12 hours, at least 18 hours, at least 24 hours, at least 1.5 days, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 18 days, at least 21 days, at least 24 days, at least 27 days, at least 30 days, at least 40 days, at least 50 days, at least 60 days, at least 70 days, at least 80 days, at least 90 days, or at least 100 days. In various embodiments, a composition of the present disclosure comprising a population of CECs (e.g., a population of cultured human CECs) and a rho kinase (ROCK) inhibitor disclosed herein, before administration, is stored for at least 6 hours, at least 9 hours, at least 12 hours, at least 18 hours, at least 24 hours, at least 1.5 days, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 18 days, at least 21 days, at least 24 days, at least 27 days, or at least 30 days. In various embodiments, a composition of the present disclosure comprising a population of CECs (e.g., a population of cultured human CECs) and a rho kinase (ROCK) inhibitor disclosed herein, before administration, is stored for at least 3 days, at least 5 days, at least 7 days, at least 9 days, or at least 10 days.

In various embodiments, a composition of the present disclosure comprising a population of CECs (e.g., a population of cultured human CECs) and a rho kinase (ROCK) inhibitor disclosed herein, before administration, is stored for about 3 days to about 7 days, about 3 days to about 8 days, about 3 days to about 9 days, about 3 days to about 10 days, about 3 days to about 11 days, about 3 days to about 12 days, about 3 days to about 13 days, about 3 days to about 14 days, about 3 days to about 15 days, about 3 days to about 18 days, about 3 days to about 21 days, about 3 days to about 24 days, about 3 days to about 27 days, about 3 days to about 30 days, about 3 days to about 40 days, about 3 days to about 50 days, about 3 days to about 60 days, about 3 days to about 70 days, about 3 days to about 80 days, about 3 days to about 90 days, about 3 days to about 100 days, about 5 days to about 7 days, about 5 days to about 8 days, about 5 days to about 9 days, about 5 days to about 10 days, about 5 days to about 11 days, about 5 days to about 12 days, about 5 days to about 13 days, about 5 days to about 14 days, about 5 days to about 15 days, about 5 days to about 18 days, about 5 days to about 21 days, about 5 days to about 24 days, about 5 days to about 27 days, about 5 days to about 30 days, about 5 days to about 40 days, about 5 days to about 50 days, about 5 days to about 60 days, about 5 days to about 70 days, about 5 days to about 80 days, about 5 days to about 90 days, about 5 days to about 100 days, about 7 days to about 9 days, about 7 days to about 10 days, about 7 days to about 11 days, about 7 days to about 12 days, about 7 days to about 13 days, about 7 days to about 14 days, about 7 days to about 15 days, about 7 days to about 18 days, about 7 days to about 21 days, about 7 days to about 24 days, about 7 days to about 27 days, about 7 days to about 30 days, about 7 days to about 40 days, about 7 days to about 50 days, about 7 days to about 60 days, about 7 days to about 70 days, about 7 days to about 80 days, about 7 days to about 90 days, or about 7 days to about 100 days. In various embodiments, a composition of the present disclosure comprising a population of CECs (e.g., a population of cultured human CECs) and a rho kinase (ROCK) inhibitor disclosed herein, before administration, is stored for about 7 days to about 9 days, about 7 days to about 10 days, about 7 days to about 11 days, about 7 days to about 12 days, about 7 days to about 13 days, about 7 days to about 14 days, about 7 days to about 15 days, about 7 days to about 16 days, about 7 days to about 17 days, about 7 days to about 18 days, about 7 days to about 19 days, or about 7 days to about 20 days.

In various embodiments, the composition is stored at a temperature disclosed herein (e.g., at a first temperature disclosed herein).

In various embodiments, a composition or a treatment or administration method disclosed herein is for administration of at least about 1×10⁵, at least about 2.5×10⁵, at least about 5×10⁵, at least about 7.5×10⁵, at least about 1×10⁶, at least about 2.5×10⁶, at least about 5×10⁶, or at least about 7.5×10⁶ CECs (e.g., cultured human CECs) to the human subject (e.g., to an eye of the subject). In various embodiments, a composition or a treatment or administration method disclosed herein is for administration of at least about 1×10⁵, at least about 5×10⁵, or at least about 1×10⁶ CECs (e.g., cultured human CECs) to the human subject (e.g., to an eye of the subject). In various embodiments, a composition or a treatment or administration method disclosed herein is for administration of at least about 1×10⁶ CECs (e.g., cultured human CECs) to the human subject (e.g., to an eye of the subject).

In various embodiments, a composition disclosed herein does not cause or substantially cause ocular adverse effects after administration. In various embodiments, the ocular adverse effects comprise ocular hypertension, conjunctival hemorrhage, epithelial defect, ocular pain, cystoid macular edema, and/or corneal abrasion. In various embodiments, “substantially cause ocular adverse effects” refers to ocular adverse effects in at least 50%, at least 55%, at least 60%, at least 65%, at least 70%, at least 75%, at least 80%, at least 85%, at least 90%, at least 95%, at least 96%, at least 97%, at least 98%, at least 99%, or 100% of subjects that receive the composition.

Corneal endothelial disease may occur, for example, when endothelial cells degrade or are lost due to conditions such as bullous keratopathy, Fuchs' dystrophy, congenital corneal dystrophies or ocular surgical trauma. Corneal endothelial disease leads to symptoms that impact vision, including blurred vision, vision loss, corneal hydration, increased glare or discomfort, or severe ocular pain.

Accordingly, in some embodiments, the CEC composition of the present disclosure is for use in treating a corneal endothelial disease. Non-limiting examples of corneal endothelial diseases include bullous keratopathy, corneal endothelial dystrophies (e.g., cornea guttata, Fuchs endothelial corneal dystrophy, posterior polymorphous corneal dystrophy, iridocorneal endothelial syndrome, and congenital hereditary corneal endothelial dystrophy), viral diseases (e.g., cytomegalovirus endotheliitis and herpetic endotheliitis), exfoliation syndrome, and corneal endothelial graft rejection; as well as inflammation or physical damage associated with external factors, such as keratouveitis, interstitial keratitis, corneal endotheliitis, corneal endothelial cell loss after corneal transplantation, corneal injury after intraocular surgery (e.g., cataract surgery, vitreous surgery, glaucoma surgery), corneal injury induced by glaucomatous attack, corneal injury caused by long-term contact lens use, corneal trauma, corneal edema, and intrapartum corneal trauma. In various embodiments, the corneal endothelial disease is selected from a bullous keratopathy, a corneal edema, a corneal leukoma, a corneal endothelial inflammation, and a corneal dystrophy; optionally wherein the corneal dystrophy is Fuchs' dystrophy.

Additional objects, advantages, and novel features of the present invention will become apparent to one ordinarily skilled in the art upon examination of the following examples, which are not intended to be limiting.

Additionally, each of the various embodiments and aspects of the present invention as delineated hereinabove and as claimed in the claims section below finds experimental support in the following examples.

Various embodiments and aspects of the present invention as delineated herein above and as claimed in the claims section below find experimental support in the following examples.

### EXEMPLARY EMBODIMENTS

Embodiment 1. A composition comprising: a population of cultured human corneal endothelial cells, and a rho kinase (ROCK) inhibitor, wherein the composition has a shelf-life of at least about 3 days at a first temperature.

Embodiment 2. The composition of embodiment 1, comprising Dulbecco's modified eagle medium (DMEM).

Embodiment 3. The composition of embodiment 1 or 2, wherein the ROCK inhibitor is Y-27632.

Embodiment 4. The composition of any one of embodiments 1-3, wherein the composition comprises about 10 μM to about 500 μM, about 50 μM to about 250 μM, about 80 μM to about 120 μM, or about 100 μM of the ROCK inhibitor; optionally wherein the composition comprises about 100 μM of the ROCK inhibitor.

Embodiment 5. The composition of any one of embodiments 1-4, wherein the first temperature is between about 10° C. and about 30° C.

Embodiment 6. The composition of embodiment 5, wherein the first temperature is between about 15° C. and about 25° C.

Embodiment 7. The composition of embodiment 5, wherein the first temperature is about 15° C.

Embodiment 8. The composition of embodiment 5, wherein the first temperature is about 20° C.

Embodiment 9. The composition of embodiment 5, wherein the first temperature is about 25° C.

Embodiment 10. The composition of any one of embodiments 1-9, wherein the composition has a shelf-life of at least 5 days, at least 7 days, at least 9 days, or at least 10 days at the first temperature.

Embodiment 11. The composition of embodiment 10, wherein the composition has a shelf-life of at least 7 days at the first temperature.

Embodiment 12. The composition of any one of embodiments 1-9, wherein the composition has a shelf-life of about 7 days to about 20 days at the first temperature.

Embodiment 13. The composition of any one of embodiments 1-12, wherein the shelf-life is represented by at least 70% viability of the population of cultured human corneal endothelial cells.

Embodiment 14. The composition of embodiment 13, wherein the shelf-life is represented by at least 75% viability of the population of cultured human corneal endothelial cells.

Embodiment 15. The composition of embodiment 13, wherein the shelf-life is represented by at least 80% viability of the population of cultured human corneal endothelial cells.

Embodiment 16. The composition of embodiment 1, wherein the composition comprises about 100 μM of Y-27632, the composition has a shelf-life of at least 5 days at the first temperature, the first temperature is between about 15° C. and about 25° C., and the shelf-life is represented by at least 70% viability of the population of cultured human corneal endothelial cells; optionally wherein the first temperature is about 15° C.; further optionally wherein the composition has a shelf-life of at least 7 days at the first temperature.

Embodiment 17. The composition of any one of embodiments 1-16, wherein at least 65%, at least 70%, at least 75%, or at least 80% of the population of cultured human corneal endothelial cells are CD166 positive, CD105 negative to weakly positive, and CD44 negative to weakly positive; optionally wherein at least 75% of the population of cultured human corneal endothelial cells are CD166 positive, CD105 negative to weakly positive, and CD44 negative to weakly positive.

Embodiment 18. The composition of any one of embodiments 1-16, wherein at least 65%, at least 70%, at least 75%, or at least 80% of the population of cultured human corneal endothelial cells are CD166 positive, CD105 negative, and CD44 negative; optionally wherein at least 75% of the population of cultured human corneal endothelial cells are CD166 positive, CD105 negative, and CD44 negative.

Embodiment 19. The composition of any one of embodiments 1-16, wherein at least 65%, at least 70%, at least 75%, or at least 80% of the population of cultured human corneal endothelial cells are CD24 negative, CD166 positive, CD105 negative to weakly positive, and CD44 negative to weakly positive; optionally wherein at least 75% of the population of cultured human corneal endothelial cells are CD24 negative, CD166 positive, CD105 negative to weakly positive, and CD44 negative to weakly positive.

Embodiment 20. The composition of any one of embodiments 1-16, wherein at least 65%, at least 70%, at least 75%, or at least 80% of the population of cultured human corneal endothelial cells are CD24 negative, CD166 positive, CD105 negative, and CD44 negative; optionally wherein at least 75% of the population of cultured human corneal endothelial cells are CD24 negative, CD166 positive, CD105 negative, and CD44 negative.

Embodiment 21. The composition of any one of embodiments 1-16, wherein at least 65%, at least 70%, at least 75%, or at least 80% of the population of cultured human corneal endothelial cells are CD26 negative, CD166 positive, CD105 negative to weakly positive, and CD44 negative to weakly positive; optionally wherein at least 75% of the population of cultured human corneal endothelial cells are CD26 negative, CD166 positive, CD105 negative to weakly positive, and CD44 negative to weakly positive.

Embodiment 22. The composition of any one of embodiments 1-16, wherein at least 65%, at least 70%, at least 75%, or at least 80% of the population of cultured human corneal endothelial cells are CD26 negative, CD166 positive, CD105 negative, and CD44 negative; optionally wherein at least 75% of the population of cultured human corneal endothelial cells are CD26 negative, CD166 positive, CD105 negative, and CD44 negative.

Embodiment 23. The composition of any one of embodiments 1-22, wherein the population of cultured human corneal endothelial cells are allogeneic.

Embodiment 24. The composition of any one of embodiments 1-23, wherein the population of cultured human corneal endothelial cells are obtained from a donor cornea.

Embodiment 25. The composition of any one of embodiments 1-23, wherein the population of cultured human corneal endothelial cells are differentiated from corneal endothelial cell-derived cells, pluripotent stem cells, mesenchymal stem cells, corneal endothelial precursor cells, cells harvested from a corneal endothelium, or corneal endothelial precursor cells or corneal endothelial-like cells produced via a direct programing method.

Embodiment 26. The composition of embodiment 25, wherein the population of cultured human corneal endothelial cells are differentiated from pluripotent stem cells; optionally wherein the pluripotent stem cells are induced pluripotent stem cells (iPSCs).

Embodiment 27. The composition of any one of embodiments 1-26, wherein the composition comprises at least about 1×10⁵, at least about 5×10⁵, or at least about 1×10⁶ cultured human corneal endothelial cells; optionally wherein the composition comprises at least about 1×10⁶ cultured human corneal endothelial cells.

Embodiment 28. The composition of any one of embodiments 1-26, wherein the composition is for administration to a human subject with a corneal endothelial disease.

Embodiment 29. The composition of embodiment 28, wherein the composition is for administration to an anterior chamber of an eye of the human subject.

Embodiment 30. The composition of embodiment 28 or 29, wherein the composition is for administration of at least about 1×10⁵, at least about 5×10⁵, or at least about 1×10⁶ cultured human corneal endothelial cells to the human subject; optionally wherein the composition is for administration of at least about 1×10⁶ cultured human corneal endothelial cells to the human subject.

Embodiment 31. The composition of any one of embodiments 28-30, wherein the composition does not cause or substantially cause ocular adverse effects after administration; optionally wherein the ocular adverse effects comprise ocular hypertension, conjunctival hemorrhage, epithelial defect, ocular pain, cystoid macular edema, and/or corneal abrasion.

Embodiment 32. The composition of any one of embodiments 28-31, wherein the composition is stored for at least 3 days at the first temperature before administration.

Embodiment 33. The composition of any one of embodiments 28-32, wherein the corneal endothelial disease is selected from a bullous keratopathy, a corneal edema, a corneal leukoma, a corneal endothelial inflammation, and a corneal dystrophy; optionally wherein the corneal dystrophy is Fuchs' dystrophy.

Embodiment 34. The composition of any one of embodiments 1-33, wherein the population of the cultured human corneal endothelial cells is in a suspension form or is not substantially adherent to a surface of a container of the composition.

Embodiment 35. A method of improving shelf-life of a composition comprising corneal endothelial cells, the method comprising storing the composition at a first temperature, wherein the composition comprises a ROCK inhibitor.

Embodiment 36. The method of embodiment 35, wherein the composition comprises DMEM.

Embodiment 37. The method of embodiment 35 or 36, wherein the ROCK inhibitor is Y-27632.

Embodiment 38. The method of any one of embodiments 35-37, wherein the composition comprises about 10 μM to about 500 μM, about 50 μM to about 250 μM, about 80 μM to about 120 μM, or about 100 μM of the ROCK inhibitor; optionally wherein the composition comprises about 100 μM of the ROCK inhibitor.

Embodiment 39. The method of any one of embodiments 35-38, wherein the first temperature is between about 10° C. and about 30° C.

Embodiment 40. The method of embodiment 39, wherein the first temperature is between about 15° C. and about 25° C.

Embodiment 41. The method of embodiment 39, wherein the first temperature is about 15° C.

Embodiment 42. The method of embodiment 39, wherein the first temperature is about 20° C.

Embodiment 43. The method of embodiment 39, wherein the first temperature is about 25° C.

Embodiment 44. The method of any one of embodiments 35-43, wherein the composition has a shelf-life of at least 5 days, at least 7 days, at least 9 days, or at least 10 days at the first temperature.

Embodiment 45. The method of embodiment 44, wherein the composition has a shelf-life of at least 7 days at the first temperature.

Embodiment 46. The method of any one of embodiments 35-43, wherein the composition has a shelf-life of about 7 days to about 20 days at the first temperature.

Embodiment 47. The method of any one of embodiments 35-46, wherein the shelf-life is represented by at least 70% viability of the corneal endothelial cells.

Embodiment 48. The method of embodiment 47, wherein the shelf-life is represented by at least 75% viability of the corneal endothelial cells.

Embodiment 49. The method of embodiment 47, wherein the shelf-life is represented by at least 80% viability of the corneal endothelial cells.

Embodiment 50. The method of embodiment 35, wherein the composition comprises about 100 μM of Y-27632, the composition has a shelf-life of at least 5 days at the first temperature, the first temperature is between about 15° C. and about 25° C., and the shelf-life is represented by at least 70% viability of the corneal endothelial cells; optionally wherein the first temperature is about 15° C.; further optionally wherein the composition has a shelf-life of at least 7 days at the first temperature.

Embodiment 51. The method of any one of embodiments 35-50, wherein at least 65%, at least 70%, at least 75%, or at least 80% of the corneal endothelial cells are CD166 positive, CD105 negative to weakly positive, and CD44 negative to weakly positive; optionally wherein at least 75% of the corneal endothelial cells are CD166 positive, CD105 negative to weakly positive, and CD44 negative to weakly positive.

Embodiment 52. The method of any one of embodiments 35-50, wherein at least 65%, at least 70%, at least 75%, or at least 80% of the corneal endothelial cells are CD166 positive, CD105 negative, and CD44 negative; optionally wherein at least 75% of the corneal endothelial cells are CD166 positive, CD105 negative, and CD44 negative.

Embodiment 53. The method of any one of embodiments 35-50, wherein at least 65%, at least 70%, at least 75%, or at least 80% of the corneal endothelial cells are CD24 negative, CD166 positive, CD105 negative to weakly positive, and CD44 negative to weakly positive; optionally wherein at least 75% of the corneal endothelial cells are CD24 negative, CD166 positive, CD105 negative to weakly positive, and CD44 negative to weakly positive.

Embodiment 54. The method of any one of embodiments 35-50, wherein at least 65%, at least 70%, at least 75%, or at least 80% of the corneal endothelial cells are CD24 negative, CD166 positive, CD105 negative, and CD44 negative; optionally wherein at least 75% of the corneal endothelial cells are CD24 negative, CD166 positive, CD105 negative, and CD44 negative.

Embodiment 55. The method of any one of embodiments 35-50, wherein at least 65%, at least 70%, at least 75%, or at least 80% of the corneal endothelial cells are CD26 negative, CD166 positive, CD105 negative to weakly positive, and CD44 negative to weakly positive; optionally wherein at least 75% of the corneal endothelial cells are CD26 negative, CD166 positive, CD105 negative to weakly positive, and CD44 negative to weakly positive.

Embodiment 56. The method of any one of embodiments 35-50, wherein at least 65%, at least 70%, at least 75%, or at least 80% of the corneal endothelial cells are CD26 negative, CD166 positive, CD105 negative, and CD44 negative; optionally wherein at least 75% of the corneal endothelial cells are CD26 negative, CD166 positive, CD105 negative, and CD44 negative.

Embodiment 57. The method of any one of embodiments 35-56, wherein the corneal endothelial cells are human.

Embodiment 58. The method of any one of embodiments 35-57, wherein the corneal endothelial cells are cultured prior to storing.

Embodiment 59. The method of any one of embodiments 35-58, wherein the corneal endothelial cells are allogeneic.

Embodiment 60. The method of any one of embodiments 34-59, wherein the corneal endothelial cells are obtained from a donor cornea.

Embodiment 61. The method of any one of embodiments 34-60, wherein the corneal endothelial cells are differentiated from corneal endothelial cell-derived cells, pluripotent stem cells, mesenchymal stem cells, corneal endothelial precursor cells, cells harvested from a corneal endothelium, or corneal endothelial precursor cells or corneal endothelial-like cells produced via a direct programing method.

Embodiment 62. The method of embodiment 61, wherein the corneal endothelial cells are differentiated from pluripotent stem cells; optionally wherein the pluripotent stem cells are induced pluripotent stem cells (iPSCs).

Embodiment 63. The method of any one of embodiments 35-62, wherein the composition comprises at least about 1×10⁵, at least about 5×10⁵, or at least about 1×10⁶ corneal endothelial cells; optionally wherein the composition comprises at least about 1×10⁶ corneal endothelial cells.

Embodiment 64. The method of any one of embodiments 35-63, wherein the composition is for administration to a human subject with a corneal endothelial disease.

Embodiment 65. The method of embodiment 64, wherein the composition is for administration to an anterior chamber of an eye of the human subject.

Embodiment 66. The method of embodiment 64 or 65, wherein the composition is for administration of at least about 1×10⁵, at least about 5×10⁵, or at least about 1×10⁶ corneal endothelial cells to the human subject; optionally wherein the composition is for administration of at least about 1×10⁶ corneal endothelial cells to the human subject.

Embodiment 67. The method of any one of embodiments 64-66, wherein the corneal endothelial cells are human and are cultured prior to administration.

Embodiment 68. The method of any one of embodiments 64-67, wherein the composition does not cause or substantially cause ocular adverse effects after administration; optionally wherein the ocular adverse effects comprise ocular hypertension, conjunctival hemorrhage, epithelial defect, ocular pain, cystoid macular edema, and/or corneal abrasion.

Embodiment 69. The method of any one of embodiments 64-68, wherein the composition is stored for at least 3 days at the first temperature before administration.

Embodiment 70. The method of any one of embodiments 64-69, wherein the corneal endothelial disease is selected from a bullous keratopathy, a corneal edema, a corneal leukoma, a corneal endothelial inflammation, and a corneal dystrophy; optionally wherein the corneal dystrophy is Fuchs' dystrophy.

Embodiment 71. The method of any one of embodiments 35-70, wherein the corneal endothelial cells of the composition are in a suspension form or are not substantially adherent to a surface of a container of the composition.

Embodiment 72. A method of storing corneal endothelial cells, comprising storing the corneal endothelial cells with a ROCK inhibitor at a first temperature.

Embodiment 73. The method of embodiment 72, comprising storing the corneal endothelial cells with the ROCK inhibitor and DMEM.

Embodiment 74. The method of embodiment 72 or 73, wherein the ROCK inhibitor is Y-27632.

Embodiment 75. The method of any one of embodiments 72-74, comprising storing the corneal endothelial cells with about 10 μM to about 500 μM, about 50 μM to about 250 μM, about 80 μM to about 120 μM, or about 100 μM of the ROCK inhibitor; the method optionally comprising storing the corneal endothelial cells with about 100 μM of the ROCK inhibitor.

Embodiment 76. The method of any one of embodiments 72-75, wherein the first temperature is between about 10° C. and about 30° C.

Embodiment 77. The method of embodiment 76, wherein the first temperature is between about 15° C. and about 25° C.

Embodiment 78. The method of embodiment 76, wherein the first temperature is about 15° C.

Embodiment 79. The method of embodiment 76, wherein the first temperature is about 20° C.

Embodiment 80. The method of embodiment 76, wherein the first temperature is about 25° C.

Embodiment 81. The method of any one of embodiments 72-80, wherein the corneal endothelial cells maintain at least 70% viability after storage at the first temperature for a first duration.

Embodiment 82. The method of embodiment 81, wherein the corneal endothelial cells maintain at least 75% viability after storage at the first temperature for the first duration.

Embodiment 83. The method of embodiment 81, wherein the corneal endothelial cells maintain at least 80% viability after storage at the first temperature for the first duration.

Embodiment 84. The method of any one of embodiments 81-83, wherein at least 65%, at least 70%, at least 75%, or at least 80% of the corneal endothelial cells are CD166 positive, CD105 negative to weakly positive, and CD44 negative to weakly positive after storage at the first temperature for the first duration; optionally wherein at least 75% of the corneal endothelial cells are CD166 positive, CD105 negative to weakly positive, and CD44 negative to weakly positive after storage at the first temperature for the first duration.

Embodiment 85. The method of any one of embodiments 81-83, wherein at least 65%, at least 70%, at least 75%, or at least 80% of the corneal endothelial cells are CD166 positive, CD105 negative, and CD44 negative after storage at the first temperature for the first duration; optionally wherein at least 75% of the corneal endothelial cells are CD166 positive, CD105 negative, and CD44 negative after storage at the first temperature for the first duration.

Embodiment 86. The method of any one of embodiments 81-83, wherein at least 65%, at least 70%, at least 75%, or at least 80% of the corneal endothelial cells are CD24 negative, CD166 positive, CD105 negative to weakly positive, and CD44 negative to weakly positive after storage at the first temperature for the first duration; optionally wherein at least 75% of the corneal endothelial cells are CD24 negative, CD166 positive, CD105 negative to weakly positive, and CD44 negative to weakly positive after storage at the first temperature for the first duration.

Embodiment 87. The method of any one of embodiments 81-83, wherein at least 65%, at least 70%, at least 75%, or at least 80% of the corneal endothelial cells are CD24 negative, CD166 positive, CD105 negative, and CD44 negative after storage at the first temperature for the first duration; optionally wherein at least 75% of the corneal endothelial cells are CD24 negative, CD166 positive, CD105 negative, and CD44 negative after storage at the first temperature for the first duration.

Embodiment 88. The method of any one of embodiments 81-83, wherein at least 65%, at least 70%, at least 75%, or at least 80% of the corneal endothelial cells are CD26 negative, CD166 positive, CD105 negative to weakly positive, and CD44 negative to weakly positive after storage at the first temperature for the first duration; optionally wherein at least 75% of the corneal endothelial cells are CD26 negative, CD166 positive, CD105 negative to weakly positive, and CD44 negative to weakly positive after storage at the first temperature for the first duration.

Embodiment 89. The method of any one of embodiments 81-83, wherein at least 65%, at least 70%, at least 75%, or at least 80% of the corneal endothelial cells are CD26 negative, CD166 positive, CD105 negative, and CD44 negative after storage at the first temperature for the first duration; optionally wherein at least 75% of the corneal endothelial cells are CD26 negative, CD166 positive, CD105 negative, and CD44 negative after storage at the first temperature for the first duration.

Embodiment 90. The method of any one of embodiments 81-89, wherein the first duration is at least 3 days, at least 5 days, at least 7 days, at least 9 days, or at least 10 days.

Embodiment 91. The method of embodiment 90, wherein the first duration is at least 7 days.

Embodiment 92. The method of embodiment 90, wherein the first duration is about 7 days to about 20 days.

Embodiment 93. The method of claim 69, comprising storing the corneal endothelial cells with about 100 μM of Y-27632 at the first temperature, wherein the first temperature is between about 15° C. and about 25° C., the corneal endothelial cells maintain at least 70% viability after storage at the first temperature for at least 5 days; optionally wherein the first temperature is about 15° C.; further optionally wherein the corneal endothelial cells maintain at least 70% viability after storage at the first temperature for at least at least 7 days.

Embodiment 94. The method of any one of embodiments 72-93, wherein the corneal endothelial cells are human.

Embodiment 95. The method of any one of embodiments 72-94, wherein the corneal endothelial cells are cultured prior to storing.

Embodiment 96. The method of any one of embodiments 72-95, wherein the corneal endothelial cells are allogeneic.

Embodiment 97. The method of any one of embodiments 72-96, wherein the corneal endothelial cells are obtained from a donor cornea.

Embodiment 98. The method of any one of embodiments 72-97, wherein the corneal endothelial cells are differentiated from corneal endothelial cell-derived cells, pluripotent stem cells, mesenchymal stem cells, corneal endothelial precursor cells, cells harvested from a corneal endothelium, or corneal endothelial precursor cells or corneal endothelial-like cells produced via a direct programing method.

Embodiment 99. The method of embodiment 98, wherein the corneal endothelial cells are differentiated from pluripotent stem cells; optionally wherein the pluripotent stem cells are induced pluripotent stem cells (iPSCs).

Embodiment 100. The method of any one of embodiments 72-99, comprising storing at least about 1×10⁵, at least about 5×10⁵, or at least about 1×10⁶ corneal endothelial cells.

Embodiment 101. The method of any one of embodiments 72-100, wherein the corneal endothelial cells are stored in a composition, and the corneal endothelial cells are in a suspension form or are not substantially adherent to a surface of a container of the composition.

Embodiment 102. A method of preparing a corneal endothelial cell composition, comprising storing the corneal endothelial cell composition at a first temperature, wherein the corneal endothelial cell composition comprises corneal endothelial cells and a ROCK inhibitor.

Embodiment 103. The method of embodiment 102, wherein the composition comprises DMEM.

Embodiment 104. The method of embodiment 102 or 103, wherein the ROCK inhibitor is Y-27632.

Embodiment 105. The method of any one of embodiments 102-104, wherein the corneal endothelial cell composition comprises about 10 μM to about 500 μM, about 50 μM to about 250 μM, about 80 μM to about 120 μM, or about 100 μM of the ROCK inhibitor; optionally wherein the corneal endothelial cell composition comprises about 100 μM of the ROCK inhibitor.

Embodiment 106. The method of any one of embodiments 102-105, wherein the first temperature is between about 10° C. and about 30° C.

Embodiment 107. The method of embodiment 106, wherein the first temperature is between about 15° C. and about 25° C.

Embodiment 108. The method of embodiment 106, wherein the first temperature is about 15° C.

Embodiment 109. The method of embodiment 106, wherein the first temperature is about 20° C.

Embodiment 110. The method of embodiment 106, wherein the first temperature is about 25° C.

Embodiment 111. The method of any one of embodiments 102-110, wherein the corneal endothelial cells maintain at least 70% viability after storage at the first temperature for a first duration.

Embodiment 112. The method of embodiment 111, wherein the corneal endothelial cells maintain at least 75% viability after storage at the first temperature for the first duration.

Embodiment 113. The method of embodiment 111, wherein the corneal endothelial cells maintain at least 80% viability after storage at the first temperature for the first duration.

Embodiment 114. The method of any one of embodiments 102-113, wherein at least 65%, at least 70%, at least 75%, or at least 80% of the corneal endothelial cells are CD166 positive, CD105 negative to weakly positive, and CD44 negative to weakly positive after storage at the first temperature for the first duration; optionally wherein at least 75% of the corneal endothelial cells are CD166 positive, CD105 negative to weakly positive, and CD44 negative to weakly positive after storage at the first temperature for the first duration.

Embodiment 115. The method of any one of embodiments 102-113, wherein at least 65%, at least 70%, at least 75%, or at least 80% of the corneal endothelial cells are CD166 positive, CD105 negative, and CD44 negative after storage at the first temperature for the first duration; optionally wherein at least 75% of the corneal endothelial cells are CD166 positive, CD105 negative, and CD44 negative after storage at the first temperature for the first duration.

Embodiment 116. The method of any one of embodiments 102-113, wherein at least 65%, at least 70%, at least 75%, or at least 80% of the corneal endothelial cells are CD24 negative, CD166 positive, CD105 negative to weakly positive, and CD44 negative to weakly positive after storage at the first temperature for the first duration; optionally wherein at least 75% of the corneal endothelial cells are CD24 negative, CD166 positive, CD105 negative to weakly positive, and CD44 negative to weakly positive after storage at the first temperature for the first duration.

Embodiment 117. The method of any one of embodiments 102-113, wherein at least 65%, at least 70%, at least 75%, or at least 80% of the corneal endothelial cells are CD24 negative, CD166 positive, CD105 negative, and CD44 negative after storage at the first temperature for the first duration; optionally wherein at least 75% of the corneal endothelial cells are CD24 negative, CD166 positive, CD105 negative, and CD44 negative after storage at the first temperature for the first duration.

Embodiment 118. The method of any one of embodiments 102-113, wherein at least 65%, at least 70%, at least 75%, or at least 80% of the corneal endothelial cells are CD26 negative, CD166 positive, CD105 negative to weakly positive, and CD44 negative to weakly positive after storage at the first temperature for the first duration; optionally wherein at least 75% of the corneal endothelial cells are CD26 negative, CD166 positive, CD105 negative to weakly positive, and CD44 negative to weakly positive after storage at the first temperature for the first duration.

Embodiment 119. The method of any one of embodiments 102-113, wherein at least 65%, at least 70%, at least 75%, or at least 80% of the corneal endothelial cells are CD26 negative, CD166 positive, CD105 negative, and CD44 negative after storage at the first temperature for the first duration; optionally wherein at least 75% of the corneal endothelial cells are CD26 negative, CD166 positive, CD105 negative, and CD44 negative after storage at the first temperature for the first duration.

Embodiment 120. The method of any one of embodiments 102-119, wherein the first duration is at least 3 days, at least 5 days, at least 7 days, at least 9 days, or at least 10 days.

Embodiment 121. The method of embodiment 116, wherein the first duration is [0297] at least 7 days.

Embodiment 122. The method of embodiment 116, wherein the first duration is about 7 days to about 20 days.

Embodiment 123. The method of embodiment 102, wherein the corneal endothelial cell composition comprises about 100 μM of Y-27632, the first temperature is between about 15° C. and about 25° C., the corneal endothelial cells maintain at least 70% viability after storage at the first temperature for at least 5 days; optionally wherein the first temperature is about 15° C.; further optionally wherein the corneal endothelial cells maintain at least 70% viability after storage at the first temperature for at least at least 7 days.

Embodiment 124. The method of any one of embodiments 102-123, wherein the corneal endothelial cells are human.

Embodiment 125. The method of any one of embodiments 102-124, wherein the corneal endothelial cells are cultured prior to storing.

Embodiment 126. The method of any one of embodiments 102-125, wherein the corneal endothelial cells are allogeneic.

Embodiment 127. The method of any one of embodiments 102-126, wherein the corneal endothelial cells are obtained from a donor cornea.

Embodiment 128. The method of any one of embodiments 102-127, wherein the corneal endothelial cells are differentiated from corneal endothelial cell-derived cells, pluripotent stem cells, mesenchymal stem cells, corneal endothelial precursor cells, cells harvested from a corneal endothelium, or corneal endothelial precursor cells or corneal endothelial-like cells produced via a direct programing method.

Embodiment 129. The method of embodiment 128, wherein the corneal endothelial cells are differentiated from pluripotent stem cells; optionally wherein the pluripotent stem cells are induced pluripotent stem cells (iPSCs).

Embodiment 130. The method of any one of embodiments 102-129, wherein the corneal endothelial cell composition comprises at least about 1×10⁵, at least about 5×10⁵, or at least about 1×10⁶ corneal endothelial cells.

Embodiment 131. The method of any one of embodiments 102-130, wherein the corneal endothelial cells are in a suspension form or are not substantially adherent to a surface of a container of the corneal endothelial cell composition.

Embodiment 132. A method of treating a corneal endothelial disease in a human subject in need thereof, comprising administering a corneal endothelial cell composition to the subject, wherein the composition comprises a population of cultured human corneal endothelial cells and a ROCK inhibitor, and the composition is stored at a first temperature for a first duration of time before administration.

Embodiment 133. The method of embodiment 132, wherein the composition comprises DMEM.

Embodiment 134. The method of embodiment 132 or 133, wherein the ROCK inhibitor is Y-27632.

Embodiment 135. The method of any one of embodiments 132-134, wherein the corneal endothelial cell composition comprises about 10 μM to about 500 μM, about 50 μM to about 250 μM, about 80 μM to about 120 μM, or about 100 μM of the ROCK inhibitor; optionally wherein the corneal endothelial cell composition comprises about 100 μM of the ROCK inhibitor.

Embodiment 136. The method of any one of embodiments 132-135, wherein the first temperature is between about 10° C. and about 30° C.

Embodiment 137. The method of embodiment 136, wherein the first temperature is between about 15° C. and about 25° C.

Embodiment 138. The method of embodiment 136, wherein the first temperature is about 15° C.

Embodiment 139. The method of embodiment 136, wherein the first temperature is about 20° C.

Embodiment 140. The method of embodiment 131, wherein the first temperature is about 25° C.

Embodiment 141. The method of any one of embodiments 142-139, wherein the first duration is at least 3 days, at least 5 days, at least 7 days, at least 9 days, or at least 10 days.

Embodiment 142. The method of embodiment 141, wherein the first duration is at least 7 days.

Embodiment 143. The method of embodiment 142, wherein the first duration is about 7 days to about 20 days.

Embodiment 144. The method of any one of embodiments 132-143, wherein the cultured human corneal endothelial cells maintain at least 70% viability after storage at the first temperature for the first duration.

Embodiment 145. The method of embodiment 144, wherein the cultured human corneal endothelial cells maintain at least 75% viability after storage at the first temperature for the first duration.

Embodiment 146. The method of embodiment 144, wherein the cultured human corneal endothelial cells maintain at least 80% viability after storage at the first temperature for the first duration.

Embodiment 147. The method of any one of embodiments 132-146, wherein at least 65%, at least 70%, at least 75%, or at least 80% of the cultured human corneal endothelial cells are CD166 positive, CD105 negative to weakly positive, and CD44 negative to weakly positive after storage at the first temperature for the first duration; optionally wherein at least 75% of the cultured human corneal endothelial cells are CD166 positive, CD105 negative to weakly positive, and CD44 negative to weakly positive after storage at the first temperature for the first duration.

Embodiment 148. The method of any one of embodiments 132-146, wherein at least 65%, at least 70%, at least 75%, or at least 80% of the cultured human corneal endothelial cells are CD166 positive, CD105 negative, and CD44 negative after storage at the first temperature for the first duration; optionally wherein at least 75% of the cultured human corneal endothelial cells are CD166 positive, CD105 negative, and CD44 negative after storage at the first temperature for the first duration.

Embodiment 149. The method of any one of embodiments 132-146, wherein at least 65%, at least 70%, at least 75%, or at least 80% of the cultured human corneal endothelial cells are CD24 negative, CD166 positive, CD105 negative to weakly positive, and CD44 negative to weakly positive after storage at the first temperature for the first duration; optionally wherein at least 75% of the cultured human corneal endothelial cells are CD24 negative, CD166 positive, CD105 negative to weakly positive, and CD44 negative to weakly positive after storage at the first temperature for the first duration.

Embodiment 150. The method of any one of embodiments 132-146, wherein at least 65%, at least 70%, at least 75%, or at least 80% of the cultured human corneal endothelial cells are CD24 negative, CD166 positive, CD105 negative, and CD44 negative after storage at the first temperature for the first duration; optionally wherein at least 75% of the cultured human corneal endothelial cells are CD24 negative, CD166 positive, CD105 negative, and CD44 negative after storage at the first temperature for the first duration.

Embodiment 151. The method of any one of embodiments 132-146, wherein at least 65%, at least 70%, at least 75%, or at least 80% of the cultured human corneal endothelial cells are CD26 negative, CD166 positive, CD105 negative to weakly positive, and CD44 negative to weakly positive after storage at the first temperature for the first duration; optionally wherein at least 75% of the cultured human corneal endothelial cells are CD26 negative, CD166 positive, CD105 negative to weakly positive, and CD44 negative to weakly positive after storage at the first temperature for the first duration.

Embodiment 152. The method of any one of embodiments 132-146, wherein at least 65%, at least 70%, at least 75%, or at least 80% of the cultured human corneal endothelial cells are CD26 negative, CD166 positive, CD105 negative, and CD44 negative after storage at the first temperature for the first duration; optionally wherein at least 75% of the cultured human corneal endothelial cells are CD26 negative, CD166 positive, CD105 negative, and CD44 negative after storage at the first temperature for the first duration.

Embodiment 153. The method of embodiment 132, wherein the corneal endothelial cell composition comprises about 100 μM of Y-27632, the first temperature is between about 15° C. and about 25° C., the cultured human corneal endothelial cells maintain at least 70% viability after storage at the first temperature for at least 5 days; optionally wherein the first temperature is about 15° C.; further optionally wherein the cultured human corneal endothelial cells maintain at least 70% viability after storage at the first temperature for at least at least 7 days.

Embodiment 154. The method of any one of embodiments 132-153, wherein the corneal endothelial cells are allogeneic.

Embodiment 155. The method of any one of embodiments 132-154, wherein the corneal endothelial cells are obtained from a donor cornea.

Embodiment 156. The method of any one of embodiments 132-155, wherein the corneal endothelial cells are differentiated from corneal endothelial cell-derived cells, pluripotent stem cells, mesenchymal stem cells, corneal endothelial precursor cells, cells harvested from a corneal endothelium, or corneal endothelial precursor cells or corneal endothelial-like cells produced via a direct programing method.

Embodiment 157. The method of embodiment 156, wherein the corneal endothelial cells are differentiated from pluripotent stem cells; optionally wherein the pluripotent stem cells are induced pluripotent stem cells (iPSCs).

Embodiment 158. The method of any one of embodiments 132-157, wherein the corneal endothelial cell composition comprises at least about 1×10⁵, at least about 5×10⁵, or at least about 1×10⁶ corneal endothelial cells.

Embodiment 159. The method of any one of embodiments 132-158, comprising administering the composition to an anterior chamber of an eye of the human subject.

Embodiment 160. The method of any one of embodiments 132-159, wherein the composition does not cause or substantially cause ocular adverse effects after administration; optionally wherein the ocular adverse effects comprise ocular hypertension, conjunctival hemorrhage, epithelial defect, ocular pain, cystoid macular edema, and/or corneal abrasion.

Embodiment 161. The method of any one of embodiments 132-160, wherein the corneal endothelial disease is selected from a bullous keratopathy, a corneal edema, a corneal leukoma, a corneal endothelial inflammation, and a corneal dystrophy; optionally wherein the corneal dystrophy is Fuchs' dystrophy.

Embodiment 162. The method of any one of embodiments 132-161, wherein the population of cultured human corneal endothelial cells is in a suspension form or is not substantially adherent to a surface of a container of the corneal endothelial cell composition.

Embodiment 163. A vial comprising the composition of any one of embodiments 1-34.

Embodiment 164. A stable cell composition comprising:
a) a population of cultured human corneal endothelial cells (CECs) comprising at least 1×10\hat{ }5 CECs; and
b) a rho kinase (ROCK) inhibitor,
wherein the composition has at least 70% cell viability following 7 days of storage at 20 degrees Celsius.

Embodiment 165. The stable cell composition of embodiment 164, wherein the composition has at least 70% cell viability following 10 days of storage at 20 degrees Celsius.

Embodiment 166. The stable cell composition of embodiment 164, wherein the composition has at least 70% cell viability following 14 days of storage at 20 degrees Celsius.

Embodiment 167. The stable cell composition of embodiment 164, wherein the composition has at least 70% cell viability following 21 days of storage at 20 degrees Celsius.

Embodiment 168. The stable cell composition of embodiment 164, wherein the composition has at least 70% cell viability following 28 days or more of storage at 20 degrees Celsius.

Embodiment 169. The stable cell composition of any one of embodiments 164-168, wherein the composition has at least 75% cell viability following storage at 20 degrees Celsius.

Embodiment 170. The stable cell composition of any one of embodiments 164-168, wherein the composition has at least 80% cell viability following storage at 20 degrees Celsius.

Embodiment 171. The stable cell composition of any one of embodiments 164-170, wherein at least 75% of the CECs in the population have an E-ratio as assessed by immunophenotyping.

Embodiment 172. The stable cell composition of any one of embodiments 164-170, wherein at least 80% of the CECs in the population have an E-ratio as assessed by immunophenotyping.

Embodiment 173. The stable cell composition of any one of embodiments 164-172, wherein the population comprises at least 1×10\hat{ }6 CECs.

Embodiment 174. The stable cell composition of any one of embodiments 164-173, wherein the CECs are obtained after at least passage 3 (P3) in culture.

Embodiment 175. The stable cell composition of any one of embodiments 164-173, wherein the CECs are obtained after at least passage 4 (P4) in culture.

Embodiment 176. The stable cell composition of any one of embodiments 164-175, wherein the ROCK inhibitor is Y-27632.

Embodiment 177. The stable cell composition of embodiment 176, wherein the composition comprises about 10 μM-500 μM, 50 μM-250 μm, 80 μM-120 μM, or 100 μM of the Y-27632.

Embodiment 178. The stable cell composition of any one of embodiments 164-177, wherein the composition is acceptable for intracameral administration.

Embodiment 179. The stable cell composition of any one of embodiments 164-178, comprising an effective dose of CECs for treating or preventing corneal endothelial cell disease in a human subject in need thereof.

Embodiment 180. The stable cell composition of embodiment 179, wherein the effective dose is about 1×10\hat{ }6 CECs per eye.

Embodiment 181. A container containing the stable cell composition of any one of embodiments 164-180.

Embodiment 182. The container of embodiment 181, wherein the CECs of the composition are not substantially adherent to the surface of the container.

Embodiment 183. The container of embodiment 181 or 182, wherein the population of CECs is about 1.70×10\hat{ }6 cells to about 5×10\hat{ }6 cells.

Embodiment 184. A method of treating or preventing a corneal endothelial disease in a human subject in need thereof, said method comprising administering the stable cell composition of embodiment 179 or 180.

### EXAMPLES

The following example describes exemplary compositions comprising human corneal endothelial cells that have improved stability, including shelf-life, and are suitable for therapeutic delivery to a human patient.

### Example 1: Stable Corneal Endothelial Cell (CEC) Compositions

Corneal endothelial cells (CECs) were harvested from the cornea of a human donor and cultured through four passages (P4). Cells were harvested, suspended in a solution containing DMEM supplemented with Human Serum Albumin (e.g., 2% HSA). 1×10⁶ cells were aliquoted into containers. Paired containers were treated with or without 100 μM ROCK inhibitor Y-27632 to determine the effect on stability of the population of CECs. Containers were stored at 20 degrees Celsius for 48 hours, 7 days, and 10 days.

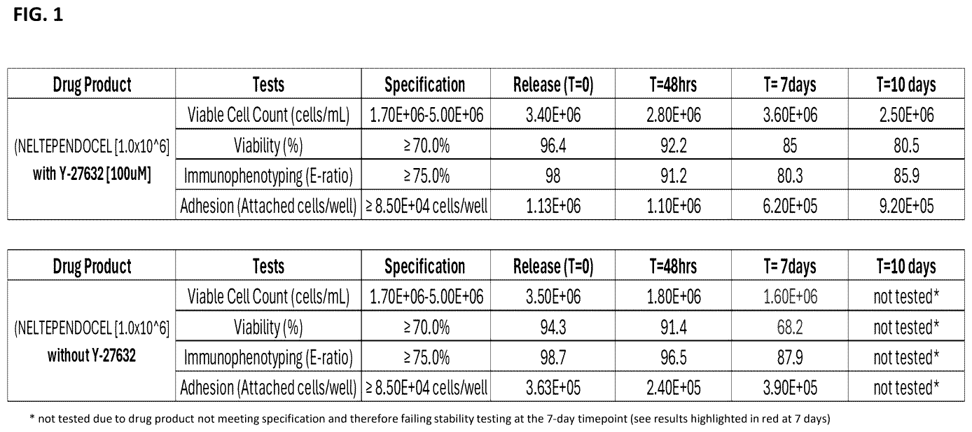

As described in FIG. 1, cells were tested at harvest and after storage for the indicated durations for cell count, viability, immunophenotyping (E-ratio), and adhesion (attached cells/well). The specification criteria and results are shown in FIG. 1. As noted, as cell count and viability were not met on Day 7 for the samples not containing Y-27632, no assays were run at Day 10 for the samples not containing Y-27632. These data demonstrate increased stability of CECs in storage in the presence of Y-27632 at the storage temperature.

CECs stored in the presence of Y-27632 at between about 10 degrees Celsius and about 30 degrees Celsius have increased stability compared to lower storage temperatures.

### Example 2: Cell Adhesion Improvement in Stable CEC Composition

The adhesion capability of CECs is an important parameter which may demonstrate the potency of CECs. The present Example demonstrates that a CEC composition formulated as disclosed herein and stored under the disclosed temperatures (e.g., a stable CEC composition) shows better cell adhesion.

Human CECs cultured and harvested as described in Example 1 were formulated with DMEM supplemented with Human Serum Albumin (e.g., 2% HSA) and 100 μM ROCK inhibitor Y-27632 into compositions. The cell compositions were stored either at a temperature between 2-8° C. or 15-25° C. After storage for 2 days at the respective temperatures, the human CECs were either directly tested for cell adhesion or loaded into syringes and held for 5, 15, 30, or 60 minutes, to mimic clinical use conditions, before testing for cell adhesion. For cell adhesion testing, the human CECs were added to and allowed to adhere to collagen I coated plates. Free, unadhered cells were decanted from the plates and adhered cells were subsequently released by enzymatic reagent TrypLE 10X. The recovered cells were counted using Chemometec NC-202 cell counter with Via-2 cassettes for evaluation of the adherent number of cells.

TABLE 1
Cell Adhesion: Total Adhered Viable Cell
(10³/well), Average ± Std Deviation

| | 15-25° C. | 2-8° C. |
|---|---|---|
| No Syringe Hold | 1520 ± 259 | 1520 ± 165 |
| 5 min Syringe Hold | 1290 ± 177 | 768 ± 322 |
| 15 min Syringe Hold | 1490 ± 115 | 782 ± 387 |
| 30 min Syringe Hold | 1320 ± 451 | 980 ± 292 |
| 60 min Syringe Hold | 1390 ± 154 | 761 ± 451 |

As shown in Table 1, larger numbers of cells adhered to collagen I coated plates from formulated CEC compositions stored at a temperature between 15-25° C. compared to those stored between 2-8° C. These data demonstrate increased cell adhesion of CECs in storage in the presence of Y-27632 at a temperature between 15-25° C.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

While specific embodiments of the disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A composition comprising a population of cultured human corneal endothelial cells and a rho kinase (ROCK) inhibitor, wherein
the population of cultured human corneal endothelia cells has at least 70% viability for at least about 3 days at a first temperature,
the first temperature is between about 10° C. and about 30° C., and
the population of the cultured human corneal endothelial cells is in a suspension form or is not substantially adherent to a surface of a container of the composition

2. The composition of claim 1, comprising Dulbecco's modified eagle medium (DMEM); optionally wherein the composition further comprises human serum albumin (HSA).

3. The composition of claim 1, wherein the ROCK inhibitor is Y-27632, optionally wherein the composition comprises about 10 μM to about 500 μM, about 50 μM to about 250 μM, about 80 μM to about 120 μM, or about 100 μM of Y-27632; further optionally wherein the composition comprises about 100 μM of Y-27632.

4. The composition of claim 1, wherein
the composition comprises about 100 μM of Y-27632,
the population of cultured human corneal endothelia cells has at least 70% viability for at least 7 days at the first temperature, and
the first temperature is between about 15° C. and about 25° C.

5. The composition of claim 1, wherein the population of cultured human corneal endothelia cells has at least 70% viability for about 7 days to about 20 days at the first temperature.

6. The composition of claim 1, wherein
(i) at least 75% of the population of cultured human corneal endothelial cells are CD166 positive, CD105 negative to weakly positive, and CD44 negative to weakly positive;
(ii) at least 75% of the population of cultured human corneal endothelial cells are CD166 positive, CD105 negative, and CD44 negative;
(iii) at least 75% of the population of cultured human corneal endothelial cells are CD24 negative, CD166 positive, CD105 negative to weakly positive, and CD44 negative to weakly positive;
(iv) at least 75% of the population of cultured human corneal endothelial cells are CD24 negative, CD166 positive, CD105 negative, and CD44 negative;
(v) at least 75% of the population of cultured human corneal endothelial cells are CD26 negative, CD166 positive, CD105 negative to weakly positive, and CD44 negative to weakly positive; or
(vi) at least 75% of the population of cultured human corneal endothelial cells are CD26 negative, CD166 positive, CD105 negative, and CD44 negative.

7. The composition of claim 1, wherein the composition comprises at least about 1×10⁵, at least about 5×10⁵, or at least about 1×10⁶ cultured human corneal endothelial cells; optionally wherein the composition comprises at least about 1.7×10⁶ cultured human corneal endothelial cells.

8. A method of treating a corneal endothelial disease in a human subject in need thereof, comprising administering a composition of claim 1 to the subject, wherein
the composition is stored for at least 3 days at the first temperature before administration.

9. The method of claim 8, wherein the composition is stored for about 7 days to about 20 days at the first temperature before administration.

10. The method of claim 8, wherein the cultured human corneal endothelial cells maintain at least 70% viability after storage at the first temperature.

11. The method of claim 8, comprising administering at least about 1×10⁵, at least about 5×10⁵, or at least about 1×10⁶ corneal endothelial cells to an anterior chamber of an eye of the human subject.

12. The method of claim 8, wherein the composition does not cause or substantially cause ocular adverse effects after administration; optionally wherein the ocular adverse effects comprise ocular hypertension, conjunctival hemorrhage, epithelial defect, ocular pain, cystoid macular edema, and/or corneal abrasion.

13. The method of claim 8, wherein the corneal endothelial disease is selected from a bullous keratopathy, a corneal edema, a corneal leukoma, a corneal endothelial inflammation, and a corneal dystrophy; optionally wherein the corneal dystrophy is Fuchs' dystrophy.

14. A vial comprising the composition of claim 1.

15. A method of improving shelf-life of a composition comprising corneal endothelial cells, the method comprising storing the composition at a first temperature, wherein
the composition comprises a ROCK inhibitor,
the first temperature is between about 10° C. and about 30° C.,
the corneal endothelial cells are in a suspension form or are not substantially adherent to a surface of a container of the composition, and
the corneal endothelial cells maintain at least 70% viability after storage at the first temperature for at least about 3 days.

16. A method of preparing a corneal endothelial cell composition, comprising storing the corneal endothelial cell composition at a first temperature, wherein
the corneal endothelial cell composition comprises corneal endothelial cells and a ROCK inhibitor,
the first temperature is between about 10° C. and about 30° C.,
the corneal endothelial cells are in a suspension form or are not substantially adherent to a surface of a container of the composition, and
the corneal endothelial cells maintain at least 70% viability after storage at the first temperature for at least about 3 days.

17. The method of claim 15, wherein the composition comprises Dulbecco's modified eagle medium (DMEM); optionally wherein the composition further comprises human serum albumin (HSA).

18. The method of claim 15, wherein the ROCK inhibitor is Y-27632, optionally wherein the composition comprises about 10 μM to about 500 μM, about 50 μM to about 250 μM, about 80 μM to about 120 μM, or about 100 μM of Y-27632; further optionally wherein the composition comprises about 100 μM of Y-27632.

19. The method of claim 15, wherein
the composition comprises about 100 μM of Y-27632,
the corneal endothelial cells maintain at least 70% viability after storage at the first temperature for at least about 7 days, and
the first temperature is between about 15° C. and about 25° C.

20. The method of claim 15, wherein
(i) at least 75% of the corneal endothelial cells are CD166 positive, CD105 negative to weakly positive, and CD44 negative to weakly positive;
(ii) at least 75% of the corneal endothelial cells are CD166 positive, CD105 negative, and CD44 negative;
(iii) at least 75% of the corneal endothelial cells are CD24 negative, CD166 positive, CD105 negative to weakly positive, and CD44 negative to weakly positive;
(iv) at least 75% of the corneal endothelial cells are CD24 negative, CD166 positive, CD105 negative, and CD44 negative;
(v) at least 75% of the corneal endothelial cells are CD26 negative, CD166 positive, CD105 negative to weakly positive, and CD44 negative to weakly positive; or
(vi) at least 75% of the corneal endothelial cells are CD26 negative, CD166 positive, CD105 negative, and CD44 negative.

21-22. (canceled)
